(19) United States
(12) Patent Application Publication
(10) Pub. No.: US 2008/0306124 A1
(43) Pub. Date: Dec. 11, 2008

(54) Polycyclic Oxadiazoles or I Soxazoles and Their Use as Sip Receptor Ligands

(76) Inventors: Rainer Albert, Basel (CH); Sven Weiler, Lorrach (DE); Frederic Zecri, Bartenheim (FR)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 11/916,610

(22) PCT Filed: Jun. 7, 2006
(86) PCT No.: PCT/EP06/05422

§ 371 Date: Jul. 3, 2008

(30) Foreign Application Priority Data
Jun. 8, 2005 (GB) 0511684.3
Dec. 8, 2005 (GB) 0525064.2
Jan. 10, 2006 (GB) 0600405.5

(51) Int. Cl. A61K 31/4245; C07D 271/06; C07D 271/10; C07D 261/06; A61K 31/42; A61P 37/02; A61P 37/08; A61P 9/00; A61P 35/00; A61P 25/00

(52) U.S. Cl. 514/364; 548/131; 548/145; 548/247; 514/378

(57) ABSTRACT

Disclosed are polycyclic compounds of formula I

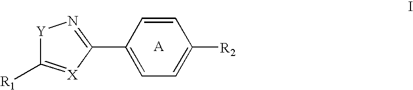

wherein R₁, R₂, X, Y and cycle A are as defined in claim 1, which have interesting pharmaceutical properties.

The present invention relates to polycyclic compounds, processes for their production, their use as pharmaceuticals and to pharmaceutical compositions comprising them.

More particularly the present invention provides in a first aspect a compound of formula I

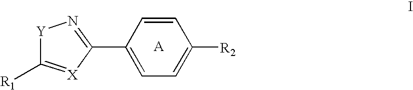

wherein
either X is —N═ and Y is —O—; or X is —O— and Y is —N═; or X is CH and Y is O;
R₁ is substituted biphenylyl, 4-phenoxy-phenyl or 4-(phenyl-C₁₋₄alkoxy)-phenyl wherein at least one of the phenyl groups is monosubstituted; phenyl substituted by one or more substituents selected from halogen, nitrile, C₁₋₈alkyl, haloC₁₋₈alkyl, C₁₋₈alkoxy, haloC_{alkoxy, —C}₁₋₈alkoxy-C₁₋₈alkoxy, C₁₋₈alkyl-C₁₋₈alkoxy, C₁₋₈alkyl-haloC₁₋₈alkoxy, haloC₁₋₈alkyl-C₁₋₈alkoxy, haloC₁₋₈alkyl-haloC₁₋₈alkoxy, haloC₁₋₈alkoxy-C₁₋₈alkoxy, C₁₋₈alkoxy-haloC₁₋₈alkoxy, haloC₁₋₈alkoxy-haloC₁₋₈alkoxy, C₁₋₈alkoxy-C₂₋₆alkyl, haloC₂₋₆alkoxy-C₃₋₆alkyl, C₃₋₆alkoxy-haloC₁₋₄alkyl, haloC₁₋₈alkoxy-haloC₁₋₈alkyl, C₂₋₆alkenyloxy, C₂₋₆alkynyloxy, C₃₋₆cycloalkyl, C₃₋₆cycloalkyl-C₁₋₄alkyl, C₃₋₆cycloalkyl-C₁₋₄alkoxy, C₃₋₆cycloalkyl-oxy, phenyl-C₁₋₄alkoxy and heterocyclic-C₁₋₄alkoxy; or substituted 5 or 6-membered heteroaryl;
R₂ is C₁₋₆ alkyl optionally substituted by halogen, OH, NH₂, C₁₋₄alkoxy or C₁₋₄alkylcarbonyloxy; amino; carboxy; sulfamoyl; carbamoyl; or HN—CO—C₁₋₄alkyl; or

### R₂ is R₃—R₄—COOH or R₃—R₄—CONR₅R₈

wherein R₃ is SO₂—NH; SO₂—N(C₁₋₄alkyl); CO—NH; CO—N(C₁₋₄alkyl); CH₂—O; NH—CO; or N(C₁₋₄alkyl)CO; and R₄ is C₁₋₆alkylene optionally interrupted by O, S or C═CH₂ or optionally substituted phenylene or C₃₋₆cycloalkylene; and each of R₅ and R₆, independently, is hydrogen or C₁₋₆alkyl or R₅ and R₆ together with the nitrogen atom to which they are bound form a heterocyclic residue, and
ring A may optionally be substituted,
provided that when Y is O, X is —N═ or —CH═ and R₂ is SO₂H—R₄—COOH wherein R₄ is branched C₁₋₆alkylene, then
i. R₁ is other than phenyl either monosubstituted by halogen, C₁₋₈alkyl, C₁₋₈alkoxy, haloC₁₋₈alkyl or haloC₁₋₈alkoxy, or disubstituted by one or two substituents selected from halogen, C₁₋₈alkyl and C₁₋₈alkoxy; or
ii. R₁ is other than monosubstituted thienyl or furyl
or a physiologically hydrolysable derivative thereof, a salt, hydrate and/or solvate thereof.

Halogen may be fluorine, chlorine or bromine, preferably fluorine or chlorine. Alkyl or alkoxy as a group or present in a group may be straight or branched. C₁₋₆alkylene may be straight or branched.

HaloC₁₋₈alkyl or haloC₁₋₈alkoxy as a group or a moiety present in a group may be C₁₋₈alkyl or C₁₋₈alkoxy substituted by 1 to 5 halogen, e.g. CF₃ or CF₃—CH₂—O—. C₁₋₈alkyl-haloC₁₋₈alkoxy may be haloC₁₋₈alkoxy further substituted by C₁₋₈alkyl, e.g. in position 1. The same may apply to the other groups.

When R₁ is substituted biphenylyl, 4-phenoxy-phenyl or 4-(phenyl-C₁₋₄alkoxy)-phenyl, either one and/or both phenyl moieties may be substituted, e.g. mono- or di-substituted e.g. by halogen, C₁₋₈alkyl, C₁₋₈alkoxy, haloC₁₋₈alkyl, haloC₁₋₈alkoxy or nitrite. Preferably at least one phenyl moiety of the biphenylyl, 4-phenoxy-phenyl or 4-(phenyl-C₁₋₄alkoxy)-phenyl is monosubstituted, e.g. as indicated above. Alternatively each phenyl moiety of the biphenylyl, 4-phenoxy-phenyl or 4-(phenyl-C₁₋₄alkoxy)-phenyl is monosubstituted, e.g. as indicated above, e.g. by haloC₁₋₈alkyl, and optionally as substituted on the second phenyl moiety either halogen, C₁₋₈alkyl or C₁₋₈alkoxy, haloC₁₋₈alkyl or haloC₁₋₈alkoxy.

When R₁ is substituted phenyl, it may be mono- or di-substituted. When is disubstituted phenyl, one substituent may preferably be haloC₁₋₈alkyl or haloC₁₋₈alkoxy and the second substituted as indicated above.

Examples of a 5 or 6-membered heteroaryl as R₁ include e.g. thienyl, furyl or pyridyl. Preferred is thienyl. When R₁ is substituted heteroaryl, it is mono- or disubstituted, preferably disubstituted. The substituent(s) may be e.g. halogen, haloC₁₋₈alkyl, e.g. CF₃, C₁₋₈alkoxy, haloC₁₋₈alkoxy, C₁₋₈alkyl, haloC₁₋₈alkyl, C₃₋₆cycloalkyl-C₁₋₄alkoxy, C₃₋₆cycloalkyl-C₁₋₄alkyl and/or phenyl optionally substituted by halogen, C₁₋₄alkyl or C₁₋₄alkoxy,

By heterocyclic residue as formed by NR₅R₆ is meant a three to eight, preferably five to eight, membered saturated, unsaturated or aromatic heterocyclic ring comprising 1 or 2 heteroatoms, preferably selected from N, O and S, and optionally substituted.

When R₂ is C₁₋₆alkyl optionally substituted by halogen, OH, NH₂, C₁₋₄alkoxy or C₁₋₄alkylcarbonyloxy, the substituent is preferably on the last carbon atom, i.e. the ω-position.

When R₄ is optionally substituted phenylene or C₃₋₆cycloalkylene, it may be 1,4-phenylene or C₃₋₆cycloalkylene, e.g. cyclohexylene, optionally substituted by halogen.

Ring A may optionally be substituted, e.g. by halogen, C₁₋₄alkyl, haloC₁₋₄alkyl, C₁₋₄alkoxy, haloC₁₋₄alkoxy or nitrile.

The following significances are preferred independently, collectively or in any combination or sub-combination:
i) R₁ is biphenylyl, 4-phenoxy-phenyl or 4-(phenyl-C₁₋₄alkoxy)-phenyl wherein at least one of the phenyl groups bears a haloC₁₋₄alkyl or haloC₁₋₈alkoxy, e.g. CF₃;
ii) R₁ is phenyl substituted by haloC₁₋₄alkyl or haloC₁₋₈alkoxy, e.g. CF₃, and by a second substituent as indicated above;
iii) R₁ is thienyl disubstituted by haloC₁₋₄alkyl or haloC₁₋₈alkoxy, e.g. CF₃, and phenyl;
iv) R₂ is SO₂NH₂;
v) R₂ is C₁₋₆alkyl (c)-substituted by NH₂, wherein R₂ is branched or straight C₁₋₆alkyl. e.g. C₁₋₄alkyl; preferably R₂ is CH₂—NH₂;
vi) R₂ is R₃—R₄—COOH;
vii) R₂ is R₃—R₄—CONR₅R₆;
viii) R₃ is SO₂—NH; SO₂—N(C₁₋₄alkyl); NH—CO; or N(C₁₋₄-alkyl)CO;
ix) R₄ is branched or linear C₁₋₆alkylene optionally interrupted by O; preferably R₄ is linear C₁₋₆alkylene;
x) Each of R₅ and R₆ independently is H or C₁₋₂alkyl;
xi) Ring A is unsubstituted.

The compounds of formula I may exist in free form or in salt form, e.g. addition salts with e.g. organic or inorganic acids, for example, hydrochloric acid or acetic acid, or salts obtainable when R₂ is or comprises COOH, with a base, e.g. alkali salts such as sodium or potassium, or substituted or unsubstituted ammonium salts.

It will be appreciated that the compounds of formula I may exist in the form of optical isomers, racemates or diastereoisomers. For example, R₄, may comprise an asymmetric carbon atom when R₄ is branched alkylene. It is to be understood that the present invention embraces all enantiomers and conformers and their mixtures. Similar considerations apply in relation to starting materials exhibiting asymmetric carbon atoms as mentioned above.

By a physiologically hydrolysable derivative of a compound of formula I is meant a compound which is hydrolysable under physiological conditions to yield a compound of formula I and a by-product which is itself physiologically acceptable, e.g. an ester which is hydrolyzed to yield a compound of formula I and a non-toxic alcohol at the desired dosage levels.

The present invention also includes a process for the production of a compound of formula I, which process comprises
a) for the production of a compound of formula I wherein X is —N═ and Y is O and R₂ is as defined above,
   reacting a compound of formula II

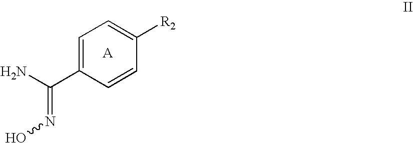

wherein ring A and R₂ are as defined above
with a compound of formula III

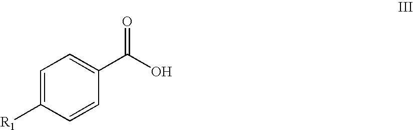

wherein R₁ is as defined above or a functional derivative thereof, e.g. an activated ester, acyl chloride or anhydride; or
b) for the production of a compound of formula I wherein X is —N═ and Y is O and R₂ is R₃—R₄—COOH or R₃—R₄—CONR₅R₆ wherein R₃ is NH—CO or N(C₁₋₄alkyl)CO and R₄, R₅ and R₆ is as defined above, reacting a compound of formula IV

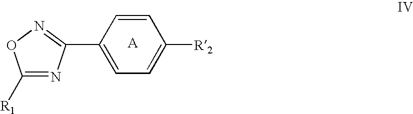

wherein R₁ and ring A are as defined above and R′₂ is NH₂ or NH(C₁₋₄alkyl), with an acylating agent or by following a Sandmeyer reaction; or
c) for the production of a compound of formula I wherein Y is —N═ and X is O, cyclizing in the presence of e.g. Burgess reagent, a compound of formula V

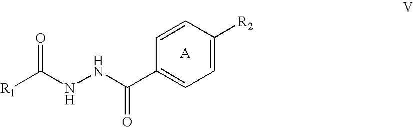

wherein R₁, R₂ and ring A are as defined above; or
d) for the production of a compound of formula I wherein Y is O and X is CH, reacting a compound of formula VI

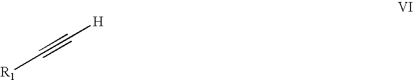

wherein R₁ is as defined above, with a compound of formula VII

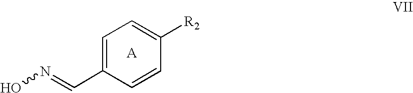

wherein R₂ is as defined above; or
e) converting a compound of formula I into another compound of formula I,
   and recovering the resulting compound of formula I in free form or in form of a salt, and, where required converting the compound of formula I obtained in free form into the desired salt form or vice versa.

The process steps a) to e) may be performed according to methods known in the art, or as disclosed below in the Examples.

Examples of conversion of a compound of formula I into another compound of formula I may include e.g.
i) for the production of a compound of formula I wherein R₁ is substituted biphenylyl, 4-phenoxy-phenyl or 4-(phenyl-C₁₋₄alkoxy)-phenyl wherein at least one of the phenyl groups is monosubstituted, converting a compound of formula I wherein R₁ is other than substituted biphenylyl, 4-phenoxy-phenyl or 4-(phenyl-C₁₋₄alkoxy)-phenyl wherein at least one of the phenyl groups is monosubstituted, into a compound of formula I wherein R₁ is substituted biphenylyl, 4-phenoxy-phenyl or 4-(phenyl-C₁₋₄alkoxy)-phenyl wherein at least one of the phenyl groups is monosubstituted.
ii) for the production of a compound of formula I wherein R₂ is R₃—R₄—COOH, hydrolyzing a compound of formula I wherein the COOH present in R₂ is in form of a physiologically hydrolysable ester, e.g. a methyl ester.
iii) for the production of a compound of formula I wherein R₂ is R₃—R₄—CONR₅R₆, converting a compound of formula I wherein R₂ is R₃—R₄—COOH into an activated ester and reacting the activated ester with the desired amine to introduce the desired R₅ and R₆ groups.

The compound of formula II used as starting material may be obtained by reacting a compound of formula VIII

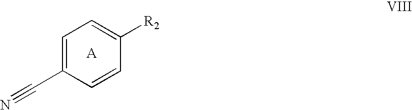

wherein R₂ and ring A are as defined above, with hydroxylamine.

The compound of formula IV may be produced by reacting a compound of formula IX

R₁—COOH IX

wherein R₁ is as defined above, or a functional derivative thereof, e.g. an activated ester, acyl chloride or anhydride, with a compound of formula X

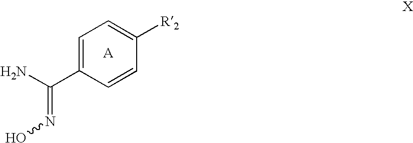

wherein R′₂ is as defined above.

Compounds of formula V may be prepared by reacting a compound of formula III with a compound of formula XI

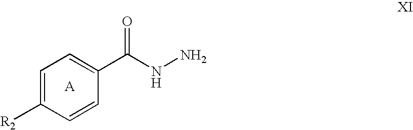

wherein R₂ and ring A are as defined above.

Insofar as the production of the starting materials is not particularly described, the compounds are either known or may be prepared analogously to methods known in the art or as disclosed hereinafter.

The following Examples are illustrative of the invention. EDC means 1-ethyl-3-(3-dimethylaminorpopyl)-carbodiimide.

### EXAMPLE 1

### 4-[5-(2-Trifluoromethyl-biphenyl-4-yl)-[1,2,4]oxadiazol-3-yl]-benzenesulfonamide

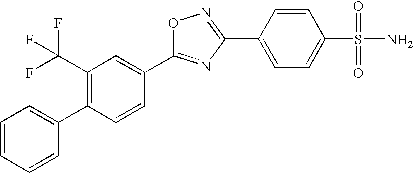

### a) 2-Trifluoromethyl-biphenyl-4-carboxylic acid

To a solution of 4-chloro-3-trifluoromethyl benzoic acid (1 eq) in THF there is added under inert atmosphere the corresponding boronic acid (1.5 eq), X-Phos (0.05 eq), KF (3 eq) and finally Pd(OAc)₂ (0.05 eq), the reaction mixture is then stirred at 90° C. for 15 hours. The reaction mixture is concentrated to dryness and purified using flash chromatography to afford the title compound.

### b) N-Hydroxy-4-sulfamoyl-benzamidine

To a solution of 4-sulfamido benzonitrile (1 eq) in THF there is added at −25° C. (ice/methanol bath) a solution of hydroxylamine in water (20 eq). The reaction mixture is then stirred at room temperature for 16 hours. The reaction mixture is extracted with ethyl acetate and washed with water, the organic layer is dried over Na₂SO₄, filtered and concentrated under reduced pressure. N-Hydroxy-4-sulfamoyl-benzamidine is isolated by precipitation using ethyl acetate/hexanes mixture.

### c) The Title Compound is Prepared as Follows

To a solution of the compound of step a) (1 eq) in dioxane there is added under inert atmosphere EDC (1.3 eq) and HOBt (1.3 eq), the reaction mixture is then stirred at room temperature for 30 minutes. Then the N-hydroxy-sulfamoyl-benzamidine of step b) (1.3 eq) is added to the reaction mixture and stirred for 30 minutes at room temperature, followed by stirring overnight at 95° C. The reaction mixture is then concentrated to dryness and purified using flash chromatography to afford 4-[5-(2-Trifluoromethyl-biphenyl-4-yl)-[1,2,4]oxadiazol-3-yl]-benzenesulfonamide (m/z= 446 (M+H)*).

### EXAMPLE 2

### N-{4-[5-(2-Trifluoromethyl-biphenyl-4-yl)-[1,2,4]oxadiazol-3-yl]-phenyl}-succinamic acid

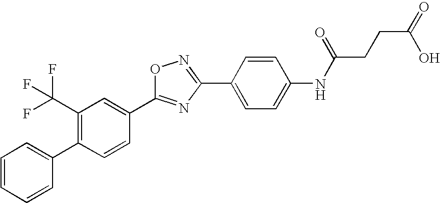

### a) 2-Trifluoromethyl-biphenyl-4-carboxylic acid

To a solution of 4-chloro-3-trifluoromethyl benzoic acid (1 eq) in THF there is added under inert atmosphere the corresponding boronic acid (1.5 eq), X-Phos (0.05 eq), KF (3 eq) and finally Pd(OAc)₂ (0.05 eq), the reaction mixture is then stirred at 90° C. for 15 hours. The reaction mixture is concentrated to dryness and purified using flash chromatography to afford the title compound.

### b) N 4-Amino-N-hydroxy-benzamidine

To a solution of 4-amino benzonitrile (1 eq) in THF there is added at −25° C. (ice/methanol 1 bath) a solution of hydroxylamine in water (20 eq). The reaction mixture is then stirred at room temperature for 16 hours. The reaction mixture is extracted with ethyl acetate and washed with water, the organic layer is dried over Na₂SO₄, filtered and concentrated under reduced pressure. 4-Amino-N-hydroxy-benzamidine is isolated by precipitation using ethyl acetate/hexanes mixture.

### c) 4-[5-(2-Trifluoromethyl-biphenyl-4-yl)-[1,2,4]oxadiazol-3-yl]-phenylamine

To a solution of the compound of step a) (1 eq) in dioxane there is added under inert atmosphere EDC (1.3 eq) and HOBt (1.3 eq), the reaction mixture is then stirred at room temperature for 30 minutes. Then the N-hydroxy-benzamidine of step b) (1.3 eq) is added to the reaction mixture and stirred for 30 minutes at room temperature, followed by stirring overnight at 95° C. The reaction mixture is then concentrated to dryness and purified using flash chromatography to afford 4-[5-(2-trifluoromethyl-biphenyl-4-yl)-[1,2,4]oxadiazol-3-yl]-phenylamine.

g) To a solution of 4-[5-(2-trifluoromethyl-biphenyl-4-yl)-[1,2,4]oxadiazol-3-yl]-phenylamine (1 eq) in acetonitrile there is added under inert atmosphere succinic anhydride (1.1 eq), and the reaction mixture is stirred at 90° C. for 16 hours. The reaction mixture is then concentrated to dryness and the desired product isolated by filtration after precipitation using a mixture of ethyl acetate and hexanes m/z= 480 (M−H)⁻.

### EXAMPLE 3

### 4-[5-(4-Phenoxy-3-trifluoromethyl-phenyl)-[1,2,4]oxadiazol-3-yl]-benzenesulfonamide

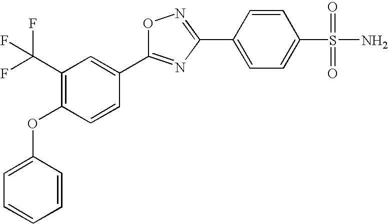

To a solution of 4-[5-(4-Fluoro-3-trifluoromethyl-phenyl)-[1,2,4]oxadiazol-3-yl]-benzene-sulfonamide obtained by following a procedure as described in Example 1 (1 eq) in DMF there is added at 0° C. (ice/water bath) under inert atmosphere phenol (3 eq) and NaH (3 eq). The reaction mixture is then stirred at room temperature for 16 hours. The reaction mixture is quenched carefully using a solution of 2N HCl and extracted with ethyl acetate, washed with water, the organic layer is dried over Na₂SO₄, filtered and concentrated under reduced pressure. The desired product is isolated by precipitation using ethyl acetate/hexanes mixture or by reverse preparative HPLC (m/z= 460 (M−H)⁻).

### EXAMPLE 4

### N-{4-[5-(4-Phenoxy-3-trifluoromethyl-phenyl)-[1,2,4]oxadiazol-3-yl]-phenyl}-succinamic acid

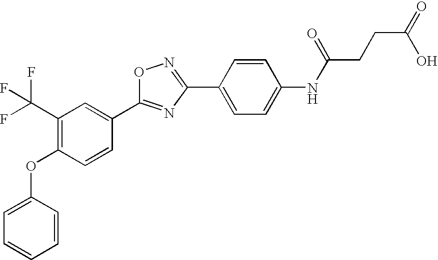

The procedure of Example 3 is repeated but using, as starting material, N-{4-[5-(4-fluoro-3-trifluoromethyl-phenyl)-[1,2,4]oxadiazol-3-yl]-phenyl}-succinamic acid to afford the title compound (m/z= 498.4 (M+H)⁺).

By following the procedure as described in Examples 1 to 4 and using the appropriate starting materials, the compounds of formula X₁

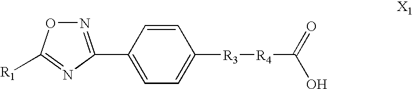

wherein R₁, R₃ and R₄ are as defined in Table 1 below, are obtained.

TABLE 1

| Example | R₁ | R₃ | R₄ | ESI+ MS: |
|---|---|---|---|---|
| 5 | 2-CF₃-4-biphenylyl | SO₂—NH | CH₂ | m/z = 502 (M − H)⁻ |
| 6 | 2-CF₃-4-biphenylyl | SO₂—NH | CH₂—CH₂ | m/z = 518 (M + H)⁺ |
| 7 | 2-CF₃-3-phenyl-5-thienyl | SO₂—NH | CH₂—CH₂ | m/z = 522 (M − H)⁻ |
| 8 | 2-CF₃-4-biphenylyl | NH—CO | CH₂—CH₂ | m/z = 480 (M − H)⁻ |
| 9 | 2-CF₃-4-biphenylyl | CO—NH | 4-Cl-1,3- | m/z = 564 (M + H)⁺ |
| | | | phenylene | |
| 10 | 2-CF₃-4-biphenylyl | CO—NH | CH₂—CH₂ | m/z = 482 (M + H)⁺ |
| 11 | 2-CF₃-3-phenyl-5-thienyl | SO₂—NH | CH₂ | m/z = 508 (M − H)⁻ |
| 12 | 2-CF₃-3-phenyl-5-thienyl | CO—NH | 4-Cl-1,3- | m/z = 570.9 (M + H)⁺ |
| | | | phenylene | |
| 13 | 2-CF₃-4-biphenylyl | CO—NH | CH₂ | m/z = 466 (M − H)⁻ |
| 14 | 2-CF₃-3-phenyl-5-thienyl | CH₂—O | 1,4-phenylene | m/z = 523.1 (M + H)⁺ |
| 15 | 2-CF₃-3-phenyl-5-thienyl | CH₂—NH | 4-Cl-1,3- | m/z = 556.1 (M + H)⁺ |
| | | | phenylene | |
| 16 | 2-CF₃-4′-fluoro-4- | SO₂—NH | CH₂ | m/z = 520 (M − H)⁻ |
| | biphenylyl | | | |
| 17 | 2-CF₃-4-(3′- | SO₂—NH | CH₂ | m/z = 536 (M − H)⁻ |
| | fluorophenoxy)-phenyl | | | |
| 18 | 2-CF₃-4-ethoxy-phenyl | SO₂—NH | CH₂ | m/z = 470 (M − H)⁻ |
| 19 | 2-CF₃-2′-fluoro-4- | SO₂—NH | CH₂ | m/z = 520 (M − H)⁻ |
| | biphenylyl | | | |
| 20 | 2-CF₃-2′-trifluoromethyl- | SO₂—NH | CH₂ | m/z = 570 (M − H)⁻ |
| | 4-biphenylyl | | | |
| 21 | 2-CF₃-4-phenoxyphenyl | SO₂—NH | CH₂CH₂ | m/z = 532 (M − H)⁻ |
| 22 | 2-CF₃-4-phenoxyphenyl | SO₂—NH | CH₂ | m/z = 518 (M − H)⁻ |
| 23 | 2-CF₃-4-benzyloxyphenyl | SO₂—NH | CH₂ | m/z = 532 (M − H)⁻ |
| 24 | 2-CF₃-4-trifluoroethoxy- | SO₂—NH | CH₂ | m/z = 524 (M − H)⁻ |
| | phenyl | | | |
| 25 | 2-CF₃-4- | SO₂—NH | CH₂CH₂ | m/z = 498 (M − H)⁻ |
| | isopropoxyphenyl | | | |
| 26 | 2-CF₃-4-(2′- | SO₂—NH | CH₂ | m/z = 536 (M − H)⁻ |
| | fluorophenoxy)-phenyl | | | |
| 27 | 2-CF₃-2′-methyl-4- | SO₂—NH | CH₂ | m/z = 516 (M − H)⁻ |
| | biphenylyl | | | |
| 28 | 2-CF₃-4- | SO₂—NH | CH₂ | m/z = 510 (M − H)⁻ |
| | cyclopentyloxyphenyl | | | |
| 29 | 2-CF₃-4-(2′- | SO₂—NH | CH₂CH₂ | m/z = 550 (M − H)⁻ |
| | fluorophenoxy)-phenyl | | | |
| 30 | 2-CF₃-2′-fluoro-4- | SO₂—NH | CH₂CH₂ | m/z = 536 (M + H)⁺ |
| | biphenylyl | | | |
| 31 | 2-CF₃-4-trifluoroethoxy- | SO₂—NH | CH₂CH₂ | m/z = 538 (M − H)⁻ |
| | phenyl | | | |
| 32 | 2-CF₃-4-(1′- | SO₂—NH | CH₂CH₂ | m/z = 552 (M − H)⁻ |
| | methyltrifluoroethoxy)- | | | |
| | phenyl | | | |
| 33 | 2-CF₃-4-(1′- | SO₂—NH | CH₂ | m/z = 510 (M − H)⁻ |
| | cyclopropylethoxy)- | | | |
| | phenyl | | | |
| 34 | 2-CF₃-4-cyclopentoxy- | SO₂—NH | CH₂CH₂ | m/z = 524 (M − H)⁻ |
| | phenyl | | | |
| 35 | 2-CF₃-4-(1′- | SO₂—NH | CH₂CH₂ | m/z = 524 (M − H)⁻ |
| | cyclopropylethoxy)- | | | |
| | phenyl | | | |
| 36 | 2-CF₃-4-biphenylyl | NH—CO | CH₂—C(CH₂) | m/z = 494.4 (M + H)⁺ |
| 37 | 2-CF₃-4-biphenylyl | NH—CO | CH₂—O—CH₂ | m/z = 496 (M − H)⁻ |
| 38 | 2-CF₃-4-biphenylyl | NH—CO | CH₂—C(CH₃)₂ | m/z = 510 (M + H)⁺ |
| 39 | 2-CF₃-4-biphenylyl | NH—CO | CH₂ | m/z = 468 (M + H)⁺ |
| 40 | 3-CF₃-4-neopentoxy- | SO₂—NH | CH(CH₃) | m/z = 526.4 (M − H)⁻ |
| | phenyl | | | |
| 41 | 3-CF₃-4-propyn-2yloxy- | SO₂—NH | CH(CH₃) | m/z = 494.3 (M − H)⁻ |
| | phenyl | | | |
| 42 | 3-CF₃-4-isobutoxy- | SO₂—NH | CH(CH₃) | m/z = 512.2 (M − H)⁻ |
| | phenyl | | | |
| 43 | 3-CF₃-4-phenoxyphenyl | SO₂—NH | C(CH₃)₂ | m/z = 548.3 (M + H)⁺ |
| 44 | 3-CF₃-4-(cyclobutyl- | SO₂—NH | CH(CH₃) | m/z = 524.2 (M − H)⁻ |
| | methoxy)-phenyl | | | |
| 45 | 2-CF₃-2′-ethyl-4- | SO₂—NH | CH₂ | m/z = 530 (M − H)⁻ |
| | biphenylyl | | | |
| 46 | 2-CF₃-2′-methyl-4- | SO₂—NH | CH₂—CH₂ | m/z = 546.3 (M − H)⁻ |
| | biphenylyl | | | |
| 47 | 2-CF₃-3′-methoxy-4- | SO₂—NH | CH₂—CH₂ | m/z = 562 (M − H)⁻ |
| | biphenylyl | | | |
| 48 | 2-CF₃-2′-chloro-4- | SO₂—NH | CH₂—CH₂ | m/z = 550 (M − H)⁻ |
| | biphenylyl | | | |
| 49 | 3-CF₃-4-isopropoxy- | SO₂—NH | CH(CH₃) | m/z = 498 (M − H)⁻ |
| | phenyl | | | |
| 50 | 3-CF₃-4-(2′,2′,2′-trifluoro- | SO₂—NH | CH₂—CH₂ | m/z = 498.4 (M + H)⁺ |
| | 1′,1′-dimethyl-ethyl)- | | | |
| | phenyl | | | |
| 51 | 2-CF₃-3′-fluoro-4- | SO₂—NH | CH₂—CH₂ | m/z = 534 (M − H)⁻ |
| | biphenylyl | | | |
| 52 | 3-CF₃-4-(cyclopropyl- | NH—CO | CH₂—CH₂ | m/z = 476.4 (M + H)⁺ |
| | methoxy)-phenyl | | | |
| 53 | 2-CF₃-2′-methyl-4- | SO₂—NH | CH₂—CH₂ | m/z = 530 (M − H)⁻ |
| | biphenylyl | | | |
| 54 | 3-CF₃-4-(3′- | SO₂—NH | CH₂—CH₂ | m/z = 550 (M − H)⁻ |
| | fluorophenoxy)-phenyl | | | |
| 55 | 2-CF₃-4-biphenylyl | NH—CO | 1,2-phenylene | m/z = 536.4 (M + H)⁺ |
| 56 | 2-CF₃-2′-CF₃-4-biphenylyl | SO₂—NH | CH₂—CH₂ | m/z = 583.9 (M − H)⁻ |
| 57 | 3-CF₃-4-(2′-methyl- | SO₂—NH | CH₂—CH₂ | m/z = 534.2 (M + H)⁺ |
| | phenoxy)-phenyl | | | |
| 58 | 3-CF₃-4-(2′-fluoro- | SO₂—NH | CH(CH₃) | m/z = 504.3 (M + H)⁺ |
| | ethoxy)-phenyl | | | |
| 59 | 3-CF₃-4-phenoxy | SO₂—NH | CH(CH₃) | m/z = 533.4 (M + H)⁺ |
| 60 | 3-CF₃-4-(2′,2′,2′-trifluoro- | SO₂—NH | CH(CH₃) | m/z = 540.2 (M + H)⁺ |
| | ethoxy)-phenyl | | | |
| 61 | 3-CF₃-4-(3′-fluoro- | SO₂—NH | CH(CH₃) | m/z = 552.1 (M + H)⁺ |
| | phenoxy)-phenyl | | | |
| 62 | 2-CF₃-2′-CF₃-4- | SO₂—NH | CH₂ | m/z = 588.3 (M + H)⁺ |
| | biphenylyl | | | |
| 63 | 3-CF₃-4-(2′-fluoro- | SO₂—NH | CH(CH₃) | m/z |
| | phenoxy)-phenyl | | | 552.1 (M + H)⁺ |
| 64 | 2-CF₃-4′-fluoro-4- | SO₂—NH | CH₂—CH₂ | m/z = 534 (M − H)⁻ |
| | biphenylyl | | | |
| 65 | 2-CF₃-4-biphenylyl | NH—CO | CH₂—CH₂—CH₂ | m/z = 496.4 (M + H)⁺ |
| 66 | 3-CF₃-4-phenoxy-phenyl | NH—CO | CH₂—CH₂ | m/z = 498.4 (M + H)⁺ |
| 67 | 3-CF₃-4-(2′-CF₃- | NH—CO | CH₂—CH₂ | m/z = 566.3 (M + H)⁺ |
| | phenoxy)-phenyl | | | |
| 68 | 3-CF₃-isopropoxy-phenyl | NH—CO | CH₂—CH₂ | m/z = 469.4 (M + H)⁺ |
| 69 | 2-CF₃-2′-Cl-4-biphenylyl | SO₂—NH | CH₂ | m/z = 536 (M − H)⁻ |
| 70 | 3-CF₃-4-(2′,2′,2′-trifluoro- | NH—CO | SO₂—NH | m/z = 504.3 (M + H)⁺ |
| | ethoxy)-phenyl | | | |
| 71 | 3-CF₃-4-(2′-methyl- | NH—CO | CH₂—CH₂ | m/z = 512.4 (M + H)⁺ |
| | phenoxy)-phenyl | | | |
| 72 | 2-CF₃-3′-fluoro-4- | SO₂—NH | CH₂ | m/z = 520 (M − H)⁻ |
| | biphenylyl | | | |
| 73 | 3-CF₃-4-(2′,2′,2′,- | SO₂—NH | CH₂ | m/z = 538 (M − H)⁻ |
| | trifluoro-1′-methyl- | | | |
| | ethoxy)-phenyl | | | |
| 74 | 3-CF₃-4-(3′-fluoro- | NH—CO | CH₂—CH₂ | m/z = 516.4 (M + H)⁺ |
| | phenoxy)-phenyl | | | |
| 75 | 2-CF₃-4-biphenylyl | NH—CO | 2,2-dimethyl- | m/z = 524.5 (M + H)⁺ |
| | | | propyl | |
| 76 | 3-CF₃-4-(2′-fluoro- | NH—CO | CH₂—CH₂ | m/z = 516.4 (M + H)⁺ |
| | phenoxy)-phenyl | | | |
| 77 | 3-CF₃-4-fluoro-phenyl | SO₂—NH | CH₂—CH₂ | m/z = 458 (M − H)⁻ |
| 78 | 3-CF₃-4-(benzyloxy)- | SO₂—NH | CH₂ | m/z = 532 (M − H)⁻ |
| | phenyl | | | |
| 79 | 3-CF₃-4-Cl-phenyl | SO₂—NH | CH₂—CH₂ | m/z = 474 (M − H)⁻ |
| 80 | 2-CF₃-2′-CF₃O-4- | SO₂—NH | CH₂—CH₂ | m/z = 586 (M − H)⁻ |
| | biphenylyl | | | |
| 81 | 3-Br-4-(phenoxy)-phenyl | SO₂—NH | CH₂—CH₂ | m/z = 545.9 (M + H)⁺ |
| 82 | 3-Br-4-(isopropoxy)- | SO₂—NH | CH₂—CH₂ | m/z = 513.1 (M + H)⁺ |
| | phenyl | | | |
| 83 | 3-Br-4-(2′-F-phenoxy)- | SO₂—NH | CH₂ | m/z = 548.4 (M + H)⁺ |
| | phenyl | | | |
| 84 | 3-Br-4-(cyclopentoxy)- | SO₂—NH | CH₂—CH₂ | m/z = 536.1 (M + H)⁺ |
| | phenyl | | | |
| 85 | 3-Br-4-(2′-F-phenoxy)- | SO₂—NH | CH₂—CH₂ | m/z = 562.4 (M + H)⁺ |
| | phenyl | | | |
| 86 | 3-Br-4-(trifluoro- | SO₂—NH | CH₂ | m/z = 536.3 (M + H)⁺ |
| | ethaneoxy)-phenyl | | | |
| 87 | 3-Br-4-(trifluoro- | SO₂—NH | CH₂—CH₂ | m/z = 550.3 (M + H)⁺ |
| | ethaneoxy)-phenyl | | | |
| 88 | 3-CN-4-(trifluoro- | SO₂—NH | CH₂ | m/z = 481.1 (M − H)⁻ |
| | ethaneoxy)-phenyl | | | |
| 89 | 3-CN-4-(phenoxy)- | SO₂—NH | CH₂ | m/z = 475.0 (M − H)⁻ |
| | phenyl | | | |

By following the procedure as described in Examples 1 to 4 and using the appropriate starting materials, the compounds of formula X₂

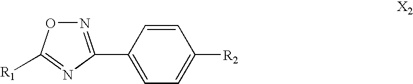

wherein R₁, and R₂ are as defined in Table 2 below, are obtained.

TABLE 2

| Example | R₁ | R₂ | ESI+ MS: |
|---|---|---|---|
| 90 | 2-CF₃-2′-fluoro-4- | SO₂—NH₂ | m/z = 462 (M − H)⁻ |
| | biphenylyl | | |
| 91 | 2-CF₃-4-biphenylyl | SO₂—NH₂ | m/z = 446 (M + H)⁺ |
| 92 | 2-CF₃-3-phenyl-5- | SO₂—NH₂ | m/z = 452.1 |
| | thienyl | | |
| 93 | 2-CF₃-2′-methyl-4- | SO₂—NH₂ | m/z = 460 (M + H)⁺ |
| | biphenylyl | | |
| 94 | 2-CF₃-3-phenyl-5- | CH₂—NH₂ | m/z = 402.1 |
| | thienyl | | |
| 95 | 2-CF₃-3-phenyl-5- | CH₂—OH | m/z = 403.1 |
| | thienyl | | |
| 96 | 2-CF₃-3-phenyl-5- | CO—NH₂ | m/z = 416.3 |
| | thienyl | | |
| 97 | 2-CF₃-4′-methyl-4- | SO₂—NH₂ | m/z = 460 (M + H)⁺ |
| | biphenylyl | | |
| 98 | 2-CF₃-3-phenyl-5- | C(CH₃)₂—OH | m/z = 430.2 |
| | thienyl | | |
| 99 | 2-CF₃-4-biphenylyl | NH—CO—CH₃ | m/z = 422 (M − H)⁻ |
| 100 | 2-CF₃-4′-fluoro-4- | SO₂—NH₂ | m/z = 464 (M + H)⁺ |
| | biphenylyl | | |
| 101 | 2-CF₃-3-phenyl-5- | CH₂—O—CO—CH₃ | m/z = 445.2 |
| | thienyl | | |
| 102 | 2-CF₃-4-biphenylyl | NH₂ | m/z = 382 (M + H)⁺ |
| 103 | 4-cyclohexyl-phenyl | SO₂—NH₂ | m/z = 384.5 (M + H)⁺ |
| 104 | 2-CF₃-3′-methoxy-4- | SO₂—NH₂ | m/z = 474 (M − H)⁻ |
| | biphenylyl | | |
| 105 | 2-CF₃-3-phenyl-5- | CH₂—F | m/z = 405.2 |
| | thienyl | | |
| 106 | 2-CF₃-4-biphenylyl | CF₃ | m/z = 435 (M + H)⁺ |
| 107 | 2-methoxy-4-biphenylyl | SO₂—NH₂ | m/z = 408 (M + H)⁺ |
| 108 | 2-CF₃-4′-methoxy-4- | SO₂—NH₂ | m/z = 474.3 (M − H)⁻ |
| | biphenylyl | | |
| 109 | 2-CF₃-3-phenyl-5- | CH₂—O—CH₃ | m/z = 417.2 |
| | thienyl | | |
| 110 | 2-CF₃-3-phenyl-5- | CH₂—CH₃ | m/z = 401.2 |
| | thienyl | | |
| 111 | 2-CF₃-3-phenyl-5- | COOH | m/z = 417.1 |
| | thienyl | | |
| 112 | 2-CF₃-4-phenoxy | SO₂—NH₂ | m/z = 460 (M − H)⁻ |
| | phenyl | | |
| 113 | 3-phenyl-5-thienyl | SO₂—NH₂ | m/z = 383 (M + H)⁺ |
| 114 | 2-CF₃-4-(2′- | SO₂—NH₂ | m/z = 430 (M − H)⁻ |
| | fluoroethoxy)phenyl | | |
| 115 | 2-CF₃-4-isopropoxy | SO₂—NH₂ | m/z = 426 (M − H)⁻ |
| | phenyl | | |
| 116 | 2-CF₃-4-trifluoroethoxy | SO₂—NH₂ | m/z = 466 (M − H)⁻ |
| | phenyl | | |
| 117 | 2-CF₃-4-2′- | SO₂—NH₂ | m/z = 478 (M − H)⁻ |
| | fluorophenoxy phenyl | | |
| 118 | 2-CF₃-4-benzyloxy | SO₂—NH₂ | m/z = 474 (M − H)⁻ |
| | phenyl | | |
| 119 | 2-fluoro-4-biphenylyl | SO₂—NH₂ | m/z = 396.4 (M + H)⁺ |
| 120 | 2-CF₃-4-(1′- | SO₂—NH₂ | m/z = 480 (M − H)⁻ |
| | methyltrifluoroethoxy)- | | |
| | phenyl | | |
| 121 | 2-CF₃-4-(1′- | SO₂—NH₂ | m/z = 440 (M − H)⁻ |
| | methylpropoxy)-phenyl | | |
| 122 | 2-CF₃-4-cyclopentoxy- | SO₂—NH₂ | m/z = 454 (M + H)⁺ |
| | phenyl | | |
| 123 | 2-CF₃-4-(1′-cyclopropyl- | SO₂—NH₂ | m/z = 454 (M + H)⁺ |
| | ethoxy)-phenyl | | |
| 124 | 3-CF₃-4-(3′-fluoro- | SO₂—NH₂ | m/z = 478.1 (M − H)⁻ |
| | phenoxy)-phenyl | | |
| 125 | 2-CF₃-2′-CF₃-4- | SO₂—NH₂ | m/z = 528.1 (M − H)⁻ |
| | biphenylyl | | |
| 126 | 3-CF₃-4-(4′-chloro- | SO₂—NH₂ | m/z = 494.1 (M − H)⁻ |
| | phenoxy)-phenyl | | |
| 127 | 3-CF₃-4-/cyclobutyl- | SO₂—NH₂ | m/z = 452.1 (M − H)⁻ |
| | methoxy)-phenyl | | |
| 128 | 3-CF₃-4-isobutoxy- | SO₂—NH₂ | m/z = 440.1 (M − H)⁻ |
| | phenyl | | |
| 129 | 3-CF₃-4-(2′-methoxy- | SO₂—NH₂ | m/z = 490.1 (M − H)⁻ |
| | phenoxy)-phenyl | | |
| 130 | 3-CF₃-4-(3′-methoxy- | SO₂—NH₂ | m/z = 490.2 (M − H)⁻ |
| | phenoxy)-phenyl | | |
| 131 | 3-CF₃-4-(2′-cyclopropyl- | SO₂—NH₂ | m/z = 452.2 (M − H)⁻ |
| | ethoxy)-phenyl | | |
| 132 | 3-CF₃-4-(propyn-2- | SO₂—NH₂ | m/z = 422.1 (M − H)⁻ |
| | yloxy)-phenyl | | |
| 133 | 3-CF₃-4-(2′-pentafluoro- | SO₂—NH₂ | m/z = 530.1 (M − H)⁻ |
| | 1′-methoxy-ethoxy)- | | |
| | phenyl | | |
| 134 | 3-CF₃-4-(cyclopropyl- | SO₂—NH₂ | m/z = 438.1 (M − H)⁻ |
| | methoxy)-phenyl | | |
| 135 | 3-CF₃-4-cyclobutoxy- | SO₂—NH₂ | m/z = 438.2 (M − H)⁻ |
| | phenyl | | |
| 136 | 3-CF₃-4-cyclohexyl-oxy- | SO₂—NH₂ | m/z = 466.2 (M − H)⁻ |
| | phenyl | | |
| 137 | 3-CF₃-4-(2′-chloro- | SO₂—NH₂ | m/z = 494.1 (M − H)⁻ |
| | phenoxy)-phenyl | | |
| 138 | 3-CF₃-4-(2′-methyl- | SO₂—NH₂ | m/z = 474.1 (M − H)⁻ |
| | phenoxy)-phenyl | | |
| 139 | 3-CF₃-4-(2′-ethoxy- | SO₂—NH₂ | m/z = 456.4 (M − H)⁻ |
| | ethoxy)-phenyl | | |
| 140 | 3-CF₃-4-(2′-methoxy-1′- | SO₂—NH₂ | m/z = 456.1 (M − H)⁻ |
| | methyl)-phenyl | | |
| 141 | 3-CF₃-4-(4′-fluoro- | SO₂—NH₂ | m/z = 478.1 (M − H)⁻ |
| | phenoxy)-phenyl | | |
| 142 | 3-CF₃-4-(5′-pyrrolidonyl- | SO₂—NH₂ | m/z = 528.1 (M − H)⁻ |
| | methoxy)-phenyl | | |
| 143 | 3-CF₃-4-neopentoxy- | SO₂—NH₂ | m/z = 454.1 (M − H)⁻ |
| | phenyl | | |
| 144 | 3-CF₃-4-(2′-2′,2′- | SO₂—NH₂ | m/z = 496.2 (M + H)⁺ |
| | trifluoro-1′,1′-dimethyl- | | |
| | ethoxy)-phenyl | | |
| 145 | 3-CF₃-4-cyclopentoxy- | SO₂—NH₂ | m/z = 454 (M + H)⁺ |
| | phenyl | | |
| 146 | 3-CF₃-4-ethoxy-phenyl | SO₂—NH₂ | m/z = 412 (M − H)⁻ |
| 147 | 3-CF₃-4-(1′-methyl- | SO₂—NH₂ | m/z = 438 (M − H)⁻ |
| | allyl)-phenyl | | |
| 148 | 3-CF₃-4-(3′-methyl- | SO₂—NH₂ | m/z = 452 (M − H)⁻ |
| | buten-2-yl)-phenyl | | |
| 149 | 3-Cl-4-(isopropyloxy)- | SO₂—NH₂ | m/z = 393.9 (M + H)⁺ |
| | phenyl | | |
| 150 | 3-Cl-4-(trifluoro- | SO₂—NH₂ | |
| | ethaneoxy)-phenyl | | |
| 151 | 3-Br-4-(cyclopropane- | SO₂—NH₂ | m/z = 450.0 (M + H)⁺ |
| | methoxy)-phenyl | | |
| 152 | 3-Br-4-(trifluoro- | SO₂—NH₂ | m/z = 478.5 (M − H)⁻ |
| | ethaneoxy)-phenyl | | |
| 153 | 3-Br-4-(3′-trifluoroiso- | SO₂—NH₂ | |
| | propyloxy)-phenyl | | |
| 154 | 3-Br-4-(1-methyl- | SO₂—NH₂ | m/z = 464.2 (M + H)⁺ |
| | cyclopropanemethoxy)- | | |
| | phenyl | | |
| 155 | 3-Br-4-(phenoxy)- | SO₂—NH₂ | m/z = 472.1 (M + H)⁺ |
| | phenyl | | |
| 156 | 3-Br-4-(cyclobutanoxy)- | SO₂—NH₂ | |
| | phenyl | | |
| 157 | (R) 3-Br-4-(butan-2- | SO₂—NH₂ | m/z = 452.4 (M + H)⁺ |
| | oxy)-phenyl | | |
| 158 | (S) 3-Br-4-(butan-2- | SO₂—NH₂ | m/z = 452.4 (M + H)⁺ |
| | oxy)-phenyl | | |
| 159 | 3-Br-4-(2′-methyl- | SO₂—NH₂ | |
| | propan-1-oxy)-phenyl | | |
| 160 | 3-Br-4-(isopropoxy)- | SO₂—NH₂ | m/z = 438.0 (M + H)⁺ |
| | phenyl | | |
| 161 | 3-Br-4-(3′-F-phenoxy)- | SO₂—NH₂ | m/z = 490.4 (M + H)⁺ |
| | phenyl | | |
| 162 | 3-Br-4-(2′-Cl-phenoxy)- | SO₂—NH₂ | m/z = 507.8 (M + H)⁺ |
| | phenyl | | |
| 163 | 3-Br-4-(2′-F-phenoxy)- | SO₂—NH₂ | m/z = 491.1 (M + H)⁺ |
| | phenyl | | |
| 164 | 3-Br-4-(isopropoxy)- | SO₂—NH₂ | m/z = 493.3 (M + H)⁺ |
| | phenyl | | |
| 165 | 3-Br-4-(2′-methyl- | SO₂—NH₂ | m/z = 486.3 (M + H)⁺ |
| | phenoxy)-phenyl | | |
| 166 | 3-CN-4-(trifluoro- | SO₂—NH₂ | m/z = 423.1 (M − H)⁻ |
| | ethaneoxy)-phenyl | | |
| 167 | 3-CN-4-(cyclopro- | SO₂—NH₂ | m/z = 395.2 (M − H)⁻ |
| | panemethoxy)-phenyl | | |
| 168 | 3-CN-4-(isopropoxy)- | SO₂—NH₂ | m/z = 383.2 (M − H)⁻ |
| | phenyl | | |
| 169 | 3-CN-4-(phenoxy)- | SO₂—NH₂ | m/z = 417.2 (M − H)⁻ |
| | phenyl | | |
| 170 | 3-CN-4-(3′- | SO₂—NH₂ | |
| | trifluoroisopropyloxy)- | | |
| | phenyl | | |
| 171 | 3-CN-4-(2′-F-phenoxy)- | SO₂—NH₂ | m/z = 435.2 (M − H)⁻ |
| | phenyl | | |
| 172 | 5-Chloro-4-isopropoxy- | SO₂—NH₂ | |
| | 3-pyridyl | | |
| 173 | 5-Chloro-4-cyclobutoxy- | SO₂—NH₂ | m/z = 406.8 (M⁺H)⁺ |
| | 3-pyridyl | | |
| 174 | 5-Chloro-4- | SO₂—NH₂ | m/z = 434.6 (M − H)⁻ |
| | trifluoroethoxy-3-pyridyl | | |
| 175 | 2-CF₃-4-biphenylyl | CH₂—NH₂ | m/z = 396.4 (M⁺H)⁺ |
| 176 | 2-CF₃-4-biphenylyl | C(CH₃)₂—NH₂ | m/z = 324.4 (M⁺H)⁺ |
| 177 | 3-CF₃-4-trifluoroethoxy- | CH₂—NH₂ | m/z = 418.3 (M⁺H)⁺ |
| | phenyl | | |
| 178 | 3-CF₃-4-isopropoxy-phenyl | CH₂—NH₂ | m/z = 378.4 (M⁺H)⁺ |

### EXAMPLE 175

The compound of Example 175 is prepared by repeating the procedure of Example 1 but using, as starting material, [4-(N-hydroxycarbamimidoyl)-benzyl]-carbamic acid tert-butyl ester (available after N-protection with BOC₂O in dioxane/water/NaOH from commercial 4-aminomethyl-benzonitrile hydrochloride) and subsequent formation of the N-hydroxy amidine with hydroxylamine 50% in water and THF as solvent) to afford the title compound after removal of the Boc-protecting group with TFA/water (95/5; 5 min., room temperature).

### EXAMPLE 176

The compound is prepared as disclosed for the compound of Example 175 but using, as starting material, 4-(1-amino-1-methyl-ethyl)-benzonitrile.

### EXAMPLE 177

To a solution of {4-[5-(4-fluoro-3-trifluoromethyl-phenyl)-[1,2,4]oxadiazol-3-yl]-benzyl}-carbamic acid tert-butyl ester obtained by following a procedure as described in Example 1 and Example 175 (1 eq) in DMF there is added at 0° C. (ice/water bath) under inert atmosphere NaH (3 eq) and after 30 minutes trifluoroethanol (5 eq) and. The reaction mixture is then stirred at room temperature for 16 hours. The reaction mixture is quenched carefully with acetic acid (95%) and concentrated. After that the residue is dissolved in methylene chloride, washed with water, the organic layer is dried over Na₂SO₄, filtered and concentrated. The desired product is obtained after purification on silica gel (cyclo-hexane/ethyle acetate 4/1 as mobile phase) and subsequent deprotection (according to Example 175).

### EXAMPLE 179

### 4-[5-(2-Trifluoromethyl-biphenyl-4-yl)-[1,3,4]oxadiazol-2-yl]-benzenesulfonamide

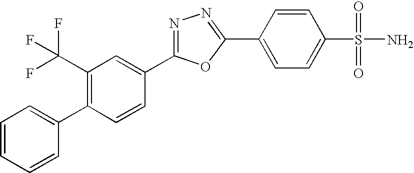

### 4-Hydrazinocarbonyl-benzenesulfonamide

To a solution of 4-sulfonamido benzoic acid (1 eq) in THF there is added under inert atmosphere Et₃N (1.3 eq) and sulfonyl chloride (1.1 eq). The reaction mixture is then stirred at room temperature for 30 minutes. The temperature is decreased to 0° C. (ice/water bath) and hydrazine in solution in methanol (30 eq) is slowly added to the reaction mixture, the resulting mixture is stirred from 0° C. to room temperature for 2 hours. The reaction mixture is quenched with water and extracted with ethyl acetate. The organic layer is dried over Na₂SO₄, filtered and concentrated under reduced pressure. The desired product is isolated by precipitation using ethyl acetate/Hexanes mixture.

To a solution of 4-phenyl-3-trifluoromethyl benzoic acid (described previously) (1 eq) in THF there is added under inert atmosphere Et₃N (1.3 eq) and sulfonyl chloride (1.1 eq), the reaction mixture is then stirred at room temperature for 5 hours. The reaction mixture is quenched with water and extracted with ethyl acetate. The organic layer is dried over Na₂SO₄, filtered and concentrated under reduced pressure. 4-[N′-(2-Trifluoromethyl-biphenyl-4-carbonyl)-hydrazinocarbonyl]-benzenesulfonamide is isolated using flash chromatography.

To a solution of 4-[N′-(2-Trifluoromethyl-biphenyl-4-carbonyl)-hydrazinocarbonyl]-benzenesulforiamide (1 eq) in acetonitrile is added under inert atmosphere Et₃N (1.3 eq) and the Burgess reagent (1.5 eq). The reaction mixture is then stirred under reflux for 10 hours. The reaction mixture is concentrated to dryness and purified using flash chromatography (ethyl acetate/hexanes) to afford the desired 4-[5-(2-Trifluoromethyl-biphenyl-4-yl)-[1,3,4]oxadiazol-2-yl]-benzenesulfonamide (m/z= 444 (M−H)⁻).

By following the procedure as described in Example 179 and using the appropriate starting materials, the compounds of formula X₃

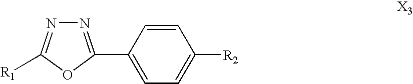

wherein R₁ and R₂ are as defined in Table 3 below, are obtained.

TABLE 3

| Example | R₁ | R₂ | ESI+ MS: |
|---|---|---|---|
| 180 | 2-CF₃-3-phenyl-5- | SO₂—NH₂ | m/z = 450 (M − H)⁻ |
| | thienyl | | |
| 181 | 2-CF₃-3-phenyl-5- | CH₂—OH | m/z = 401 (M − H)⁻ |
| | thienyl | | |
| 182 | 2-CF₃-4-biphenylyl | SO₂—NH₂ | m/z = 444 (M − H)⁻ |

### EXAMPLE 183

### 4-[5-(2-Trifluoromethyl-biphenyl-4-yl)-[1,3,4]oxadiazol-2-yl]-phenylamine

### 2-Trifluoromethyl-biphenyl-4-carboxylic acid

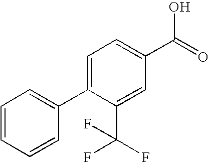

4-Chloro-3-(trifluoromethyl)-benzoic acid (1 eq), phenylboronic acid (1.8 eq), Pd(OAc)₂ and dicyclohexylphosphino-2,4,6-triisopropylbiphenyl is dissolved in THF and is refluxed for 90 minutes. After cooling the reaction mixture is filtered through Hyflo Super Cel® and concentrated. The crude residue is purified on silica gel using diethylether/c-hexane as mobile phase.

### 5-(4-Nitro-phenyl)-3-(2-trifluoromethyl-biphenyl-4-yl)-[1,2,4]oxadiazole

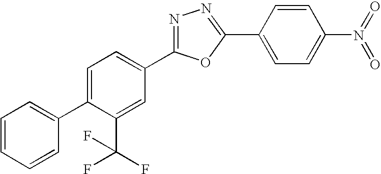

2-Trifluoromethyl-biphenyl-4-carboxylic acid (1 eq) is dissolved in POCl₃ and 4-nitrobenzhydrazide (1 eq) is added. After 3 hours at reflux another equivalent of 4-nitrobenzhydrazide is added and kept for additional 3 hours at reflux. After removal of POCl₃ under reduced pressure the residue is dissolved in ethyl acetate and extracted with saturated NaHCO₃ solution. The organic layer is dried over Na₂SO₄ and title product is used for the next step without further purification.

### 4-[5-(2-Trifluoromethyl-biphenyl-4-yl)-[1,3,4]oxadiazol-2-yl]-phenylamine

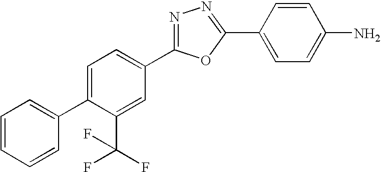

5-(4-Nitro-phenyl)-3-(2-trifluoromethyl-biphenyl-4-yl)-[1,2,4]oxadiazole is dissolved in methanol/ethyl acetate 1/1 and hydrogenated at room temperature under normal pressure with Pd/C_{10%} as catalyst for 16 hours. After filtration through Hyflo Super Cel® the reaction mixture is concentrated and purified on silica gel (methylene chloride→methylene chloride/methanol 95/5 as mobile phase).

ESI-MS (ESI): 380 (M−1H)⁻

### EXAMPLE 184

### N-{4-[5-(2-Trifluoromethyl-biphenyl-4-yl)-[1,3,4]oxadiazol-2-yl]-phenyl}-succinamic acid

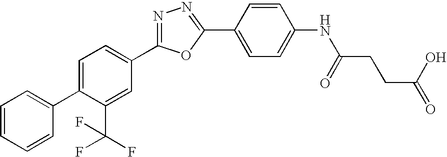

4-[5-(2-Trifluoromethyl-biphenyl-4-yl)-[1,3,4]oxadiazol-2-yl]-phenylamine (1 eq) is dissolved in methylene chloride and 4-methylmorpholine (2 eq) and succinic anhydride (2 eq) is added. After 16 hours at room temperature pure title product is obtained after silica gel column chromatography (methylene chloride→methylene chloride/methanol 90/10 as mobile phase).

ESI-MS (ESI⁻): (M−1H)⁻: 480 (M−1H)⁻

### EXAMPLE 185

### {4-[5-(2-trifluoromethyl-biphenyl-4-yl)-isoxazol-3-yl]-phenyl}-methanol

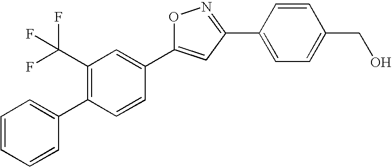

### 4-Ethynyl-2-trifluoromethyl-biphenyl

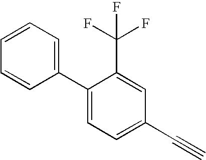

To a solution of 4-bromo-2-trifluoroaniline (1 eq) in benzene there is added under inert atmosphere n-pentylnitrite (1 eq) at 50° C. After one hour refluxing a second equivalent of n-pentytnitrite is added. After additional two hours of refluxing the reaction mixture is cooled to room temperature and concentrated under reduced pressure. The dark residue is purified on silica gel using c-hexane→c-hexane/ethyl acetate 9/1 as mobile phase (pale orange oil) to afford 4-bromo-2-trifluoromethyl-biphenyl.

4-Bromo-2-trifluoromethyl-biphenyl (1 eq) is dissolved in toluene/triethylamine (4/1) under argon gas and CuI (0.33 eq) and Pd(Ph₃P)₂Cl₂ (0.42 eq) are added and kept at 60° C. for 20 minutes. After that trimethylsilylacetylene (11.6 eq) is added dropwise to the reaction mixture. After 18 hours at 60° C. the reaction mixture is cooled down to room temperature, filtered through Hyflo Super Cel® and 3 times extracted with saturated aqueous NH₄Cl solution. The organic layer is dried with Na₂SO₄, concentrated and purified over silica gel using c-hexane as eluant yielding a pale brown liquid, trimethyl-(2-trifluoromethyl-biphenyl-4-ylethynyl)-silane.

Trimethyl-(2-trifluoromethyl-biphenyl-4-ylethynyl)-silane is dissolved in methanol/1N NaOH (4/1) and kept at room temperature for 1 hour. After removal of methanol under reduced pressure the residue is dissolved in methylene chloride and extracted with diluted aqueous HCl solution. The organic phase is dried over Na₂SO₄, filtered and concentrated. 4-Ethynyl-2-trifluoromethyl-biphenyl is obtained as a pale brown liquid.

### 4-bromo-benzaldehyde oxime

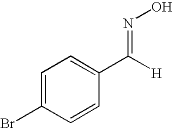

To a solution of 4-bromobenzaldehyde (1 eq) in ethanol is added at room temperature K₂CO₃ (1.1 eq) and hydroxylamine hydrochloride (1 eq). After 18 hours at room temperature the reaction mixture is filtered and concentrated under reduced pressure. The crystalline residue is dissolved in methylene chloride and extracted with diluted aqueous HCl solution. The aqueous solution is extracted with ethyl acetate and the combined organic phases are dried over Na₂SO₄, filtered and concentrated. The crystalline pale brown residue (4-bromo-benzaldehyde oxime) is used for isoxazole formation without any further purification.

### 3-(4-bromo-phenyl)-5-(2-trifluoromethyl-biphenyl-4-yl)-isoxazole

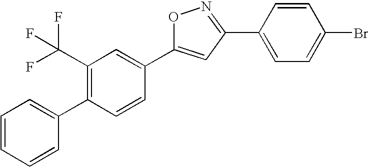

To a solution of 4-ethynyl-2-trifluoromethyl-biphenyl (1 eq) in methylene chloride a 10% aqueous solution of NaOCl is added at 0° C. After that a solution of 4-bromo-benzaldehyde oxime (1.1 eq) (b) is added and then stirred at room temperature for 1 hour. The reaction mixture is diluted with methylene chloride and 3 times extracted with water. The organic layer is dried over Na₂SO₄, filtered and concentrated. After recrystallization from methanol pure 3-(4-bromo-phenyl)-5-(2-trifluoromethyl-biphenyl-4-yl)-isoxazole (pale brown crystals) is obtained.

### {4-[5-(2-trifluoromethyl-biphenyl-4-yl)-isoxazol-3-yl]-phenyl}-methanol

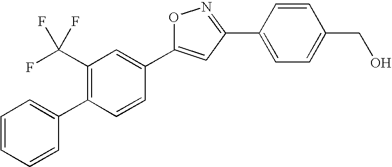

The title compound is obtained using in the procedure of 3-(4-bromo-phenyl)-5-(2-trifluoromethyl-biphenyl-4-yl)-isoxazole using 4-hydroxymethyl-benzaldehyde oxime instead of 4-bromo-benzaldehyde oxime.

ESI-MS (ESI⁺): 396 (M+1H)⁺

### EXAMPLE 186

### 4-[5-(2-trifluoromethyl-biphenyl-4-yl)-isoxazol-3-yl]-benzylamine

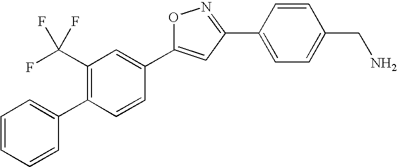

To a solution of the endproduct of Example 185 {4-[5-(2-trifluoromethyl-biphenyl-4-yl)-isoxazol-3-yl]-phenyl}-methanol (1 eq) in methylene chloride/CCl₄ ¼ sodium azide (1.2 eq) and triphenylphosphine (2.1 eq) are added. After 6 hours at reflux the reaction mixture is cooled to room temperature, quenched with water and 3 times extracted with methylene chloride. The raw material (azide) is purified on silica gel with methylene chloride as mobile phase. The purified intermediate (azide) is dissolved in methanol and hydrogenated under normal pressure with Pd/C_{10%} as catalyst until all starting material disappeared. After that the reaction mixture is filtered through Hyflo Super Cel®, concentrated and purified on silica gel using methylene chloride/methanol/acetic acid_{50%} 9/1/0.125 as eluent to afford the title compound (acetate salt) as pale yellow lyophilisate.

ESI-MS (ESI⁺): 395 (M+1H)⁺

### EXAMPLE 187

### {4-[5-(2-trifluoromethyl-biphenyl-4-yl)-isoxazol-3-yl]-benzenesulfonyl-amino}-propionic acid

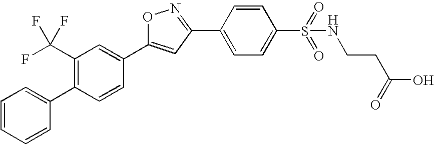

### 4-Ethynyl-2-trifluoromethyl-biphenyl

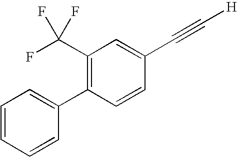

To a solution of 4-bromo-2-trifluoroaniline (1 eq) in benzene there is added under inert atmosphere n-pentylnitrite (1 eq) at 50° C. After one hour refluxing a second equivalent of n-pentylnitrite is added. After additional two hours of refluxing the reaction mixture is cooled to room temperature and concentrated under reduced pressure. The dark residue is purified on silica gel using c-hexane→c-hexane/ethyl acetate 9/1 as mobile phase (pale orange oil) to afford 4-bromo-2-trifluoromethyl-biphenyl.

4-Bromo-2-trifluoromethyl-biphenyl (1 eq) is dissolved in toluene/triethylamine (4/1) under argon gas and CuI (0.33 eq) and Pd(Ph₃P)₂Cl₂ (0.42 eq) are added and kept at 60° C. for 20 minutes. After that trimethylsilylacetylene (11.6 eq) is added dropwise to the reaction mixture. After 18 hours at 60° C. the reaction mixture is cooled down to room temperature, filtered through Hyflo Super Cel® and 3 times extracted with saturated aqueous NH₄Cl solution. The organic layer is dried with Na₂SO₄, concentrated and purified over silica gel using c-hexane as eluent yielding a pale brown liquid (trimethyl-(2-trifluoromethyl-biphenyl-4-ylethynyl)-silane).

Trimethyl-(2-trifluoromethyl-biphenyl-4-ylethynyl)-silane is dissolved in methanol/1N NaOH (4/1) and kept at room temperature for 1 hour. After removal of methanol under reduced pressure the residue is dissolved in methylene chloride and extracted with diluted aqueous HCl solution. The organic phase is dried over Na₂SO₄, filtered and concentrated. A pale brown liquid is obtained.

### 3-[4-(Hydroxyimino-methyl)-benzenesulfonylamino]-propionic acid methylester

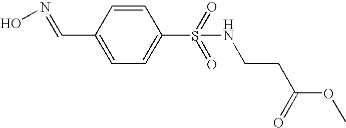

To a solution of 4-cyano-benzenesulfonyl chloride (1 eq) in dry pyridine is added at room temperature H-β-Ala-OMe (1 eq). After 1 hour at room temperature the reaction mixtures is concentrated under reduced pressure and the residue is dissolved in ethyl acetate and extracted with diluted aqueous HCl solution. The organic phase is dried over Na₂SO₄, filtered and concentrated. The honey like pale brown residue (3-(4-cyano-benzenesulfonylamino)-propionic acid methyl ester) is used for step b) without any further purification.

3-(4-Cyano-benzenesulfonylamino)-propionic acid methyl ester (1 eq) is dissolved in formic acid (75%) and Ra—Ni (FLUKA 83440; 4 eq) is added. After 3 hours at 100° C. the reaction mixture is filtered through Hyflô Super Cel® and the catalyst/Hyflo Super Cel® is washed 2 times with ethanol (with caution→flammable). The resulting solution is concentrated and is used for step 3) without any further purification.

Hydroxylamine hydrochloride (1.25 eq) is dissolved in water and NaHCO₃ (1.9 eq) is added. After 30 minutes at room temperature endproduct of step 2) dissolved in methanol is added. After 2 hours at room temperature the reaction mixture is concentrated and the residue is 3 times extracted with ethyl acetate. The combined organic phases are dried over Na₂SO₄, filtered and concentrated. After purification (flash chromatography; silica gel; methylene chloride/methanol 95/5 as mobile phase) pure title compound is isolated.

### 3-{4-[5-(2-Trifluoromethyl-biphenyl-4-yl)-isoxazol-3-yl]-enzene-sulfonylamino}-propionic acid

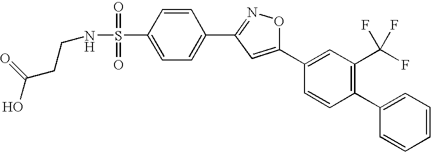

To a solution of 4-ethynyl-2-trifluoromethyl-biphenyl-(1 eq) in methylene chloride a 10% aqueous solution of NaOCl is added at 0° C. After that a solution of 3-[4-(hydroxyimino-methyl)-benzenesulfonylamino]-propionic acid methyl ester (1.1 eq) is added and then stirred at room temperature for 1 hour. The reaction mixture is diluted with methylene chloride and 3 times extracted with water. The combined organic layers are dried over Na₂SO₄, filtered and concentrated.

The resulting ester is saponified as follows:

LiOH (1.6 eq) is dissolved in methanol/water (1/1) and the ester (1 eq) is added. After 4 hours at 50° C. methanol is removed under reduced pressure, the pH is adjusted to ˜3 with 1N HCl and the reaction mixture is 3 times extracted with ethyl acetate. The combined organic layers are dried over NazSO₄, filtered, concentrated and purified on silica gel (methylene chloride/methanol 95/5 as mobile phase).

ESI-MS (ESI): 515 (M−1H)⁺

### EXAMPLE 188

### 4-[5-(2-Trifluoromethyl-biphenyl-4-yl)-isoxazol-3-yl]-phenylamine

### 3-(4-Nitro-phenyl)-5-(2-trifluoromethyl-biphenyl-4-yl)-isoxazole

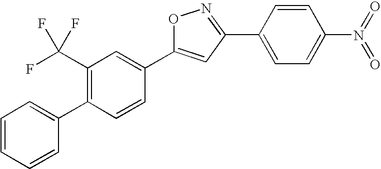

The title compound is obtained using in the procedure of 3-(4-bromo-phenyl)-5-(2-trifluoromethyl-biphenyl-4-yl)-isoxazole using 4-nitro-benzaldehyde oxime instead of 4-bromo-benzaldehyde oxime.

ESI-MS (ESI⁺): 411 (M+1H)⁺

### 4-[5-(2-Trifluoromethyl-biphenyl-4-yl)-isoxazol-3-yl]-phenylamine

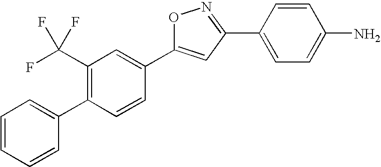

3-(4-Nitro-phenyl)-5-(2-trifluoromethyl is dissolved in methanol/ethyl acetate 1/1 and hydrogenated at room temperature under normal pressure with Pd/C_{10%} as catalyst for 16 hours. After filtration through Hyflo Super Cel® the reaction mixture is concentrated and purified on silica gel (methylene chloride→methylene chloride/methanol 95/5 as mobile phase). 4-[5-(2-Trifluoromethyl-biphenyl-4-yl)-isoxazol-3-yl]-phenylamine is isolated as a brown oil.

ESI-MS (ESI⁺): 381 (M+1H)⁺

### EXAMPLE 189

### N-{4-[5-(2-Trifluoromethyl-biphenyl-4-yl)-isoxazol-3-yl]-phenyl}-succinamic acid

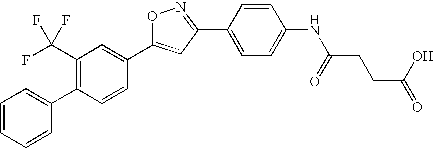

4-[5-(2-Trifluoromethyl-biphenyl-4-yl)-isoxazol-3-yl]-phenylamine is dissolved in methylene chloride and 4-methylmorpholine (2 eq) and succinic anhydride (2 eq) is added. After 16 hours at room temperature pure title product is obtained after silica gel column chromatography (methylene chloride→methylene chloride/methanol 90/10 as mobile phase).

ESI-MS (ESI⁻): (M−1H)⁻: 479 (M−1H)⁻

### EXAMPLE 190

### (R)-2-{4-[5-(2-Trifluoromethyl-biphenyl-4-yl)-isoxazol-3-yl]-benzene-sulfonylamino}-propionic acid

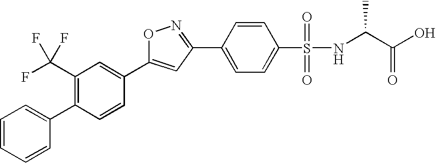

### 4-[5-(2-Trifluoromethyl-biphenyl-4-yl)-isoxazol-3-yl]-benzenesulfonyl chloride

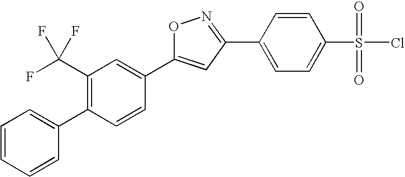

[5-(2-Trifluoromethyl-biphenyl)-isoxazol-3-yl]-phenylamine (1 eq) is dissolved in acetonitrile and after addition of HCl (conc. 37%) and an aqueous solution of NaNO₂ (1.5 eq) the reaction mixture is kept at 8° C. for 30 minutes. To this reaction a saturated solution of SO₂ in glacial acetic acid (1 ml) and subsequently a solution of CuCl₂ in water (0.5 eq) is added. After 3 hours at room temperature the precipitate is filtered off, dissolved in methylene chloride and dried over Na₂SO₄. After removal of the solvent the title compound is obtained as grey crystals.

ESI-MS (ESI⁺): 364 (M+1H)⁺

4-[5-(2-Trifluoromethyl-biphenyl-4-yl)-isoxazol-3-yl]-benzenesulfonyl chloride (1 eq) is dissolved in THF and H-DAla-OH (3 eq), triethylamine (1 eq) and 1N NaOH (2 eq) is added. After 18 hours at room temperature the reaction is diluted with water and the pH is adjusted with 1N HCl to ˜3. After extraction with methylene chloride (3 times) the organic layer is dried over Na₂SO₄, filtered and concentrated. The residue is purified on silica gel (methylene chloride/methanol/acetic acid_{50%} 9/1/0.125 as mobile phase).

ESI-MS (ESI⁺): 517 (M+1H)⁺

### EXAMPLE 191

### (S)-2-{4-[5-(2-Trifluoromethyl-biphenyl-4-yl)-isoxazol-3-yl]-benzene-sulfonylamino}-propionic acid

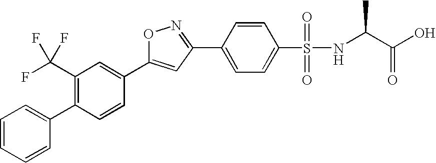

4-[5-(2-Trifluoromethyl-biphenyl-4-yl)-isoxazol-3-yl]-benzenesulfonyl chloride (1 eq) is dissolved in THF and H-Ala-OH (3 eq), triethylamine (1 eq) and 1N NaOH (2 eq) is added. After 18 hours at room temperature the reaction is diluted with water and the pH is adjusted with 1N HCl to ˜3. After extraction with methylene chloride (3 times) the organic layer is dried over Na₂SO₄, filtered and concentrated. The residue is purified on silica gel (methylene chloride/methanol/acetic acid_{50%} 9/1/0.125 as mobile phase).

ESI-MS (ESI⁺): 517 (M+1H)⁺

### EXAMPLE 192

### 4-{5-[4-(2,2,2-Trifluoro-ethoxy)-3-trifluoromethyl-phenyl]-isoxazol-3-yl}-benzenesulfonamide

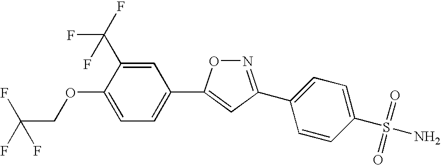

### (4-Fluoro-3-trifluoromethyl-phenylethynyl)-trimethyl-silane

The compound is synthesized according to the procedure given in: J. Org. Chem. 46(11); 1981, pp 2283

### 4-ethynyl-1-fluoro-2-trifluoromethyl-benzene

Endproduct of step 1) is reacted as given in J. Org. Chem. 46(11); 1981, pp 2283

### 4-[5-(4-fluoro-3-trifluoromethyl-phenyl)-isoxazol-3-yl]-benzenesulfonamide

Title compound is synthesized according to example 183c).

### 4-{5-[4-(2,2,2-trifluoro-ethoxy)-3-trifluoromethyl-phenyl]-isoxazol-3-yl}-benzenesulfonamide

4-[5-(4-Fluoro-3-trifluoromethyl-phenyl)-isoxazol-3-yl]-benzenesulfonamide (1 eq.) is dissolved in DMF and after addition of NaH (2 eq.; FLUKA 62863) 2,2,2-trifluoro-ethanol is added after 30 minutes at room temperature. After 3 hours at room temperature the reaction mixture is concentrated and the title compound is isolated after treatment with diethylether as pale yellow solid.

ESI-MS (ESI): 465 (M−1H)⁻

### EXAMPLE 193

### 2-Ethyl-4-[5-(2-trifluoromethyl-biphenyl-4-yl)-[1,2,4]oxadiazol-3-yl]-benzenesulfonamide

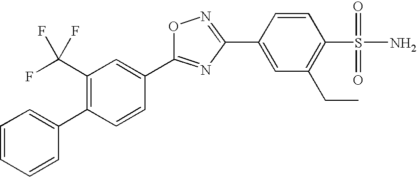

### 4-Bromo-2-ethyl-benzenesulfonamide

To a solution of 4-bromo-2-ethyl-benzenesulfonyl chloride (4.4 g, 0.015 mole) in dioxane (60 ml) was added a solution of concentrated NH₄OH (6 ml). The reaction mixture was stirred at room temperature for 2 hours. Solvent was removed under reduce pressure, and the resulting oil dissolved in ethyl acetate. The organic layer was washed with water, extracted, dried over Na₂SO₄ acid concentrated. The desired product (4 g) was isolated after crystallization using a mixture of ethyl acetate and hexanes.

### 4-Cyano-2-ethyl-benzenesulfonamide

To a solution of 4-bromo-2-ethyl-benzenesulfonamide (1.0 g, 0.004 mole) in NMP (30 ml) was added CuCN (3.6 g, 0.04 mole), the reaction mixture was stirred at 140° C. for 3 days. After allowing the reaction mixture to cool down, ethyl acetate and water were then added, the organic layer was washed with water, extracted, dried over Na₂SO₄ and concentrated. Purification using Flash column followed by crystallization yield the desired product (160 mg).

### 3-Ethyl-N-hydroxy-4-sulfamoyl-benzamidine

To a solution of 4-Cyano-2-ethyl-benzenesulfonamide (160 mg, 0.0007 mole) in THF (6 ml) was added a solution of hydroxyl amine (50% in water) (6 ml). The reaction mixture was stirred at room temperature for 16 hours. The reaction mixture was extracted with ethyl acetate, washed with water, extracted, dried over Na₂SO₄ and concentrated. The desired product (140 mg) was isolated after crystallization using a mixture of ethyl acetate and hexanes.

### 2-Trifluoromethyl-biphenyl-4-carboxylic acid

To a solution of 4-chloro-3-trifluoromethyl benzoic acid (5 g, 0.02 mole) in THF (200 ml) was added under inert atmosphere phenyl boronic acid (5.3 g, 0.04 mole), X-Phos (1 g, 0.002 mol), KF (4 g, 0.06 mole) and finally Pd(OAc)₂ (240 mg, 0.001), the reaction mixture is then stirred at 90° C. for 15 hours. The reaction mixture is concentrated to dryness and purified using flash chromatography to afford the title compound (5 g).

### 2-Ethyl-4-[5-(2-trifluoromethyl-biphenyl-4-yl)-[1,2,4]oxadiazol-3-yl]-benzenesulfonamide

To a solution of 2-trifluoromethyl-biphenyl-4-carboxylic acid. (50 mg, 0.0002 mole) in DMF (2 ml) was added under inert atmosphere EDC (40 mg, 0.0002 mole), HOBt (30 mg, 0.0002 mol), the reaction mixture was stirred at room temperature for 15 minutes, then was added 3-ethyl-N-hydroxy-4-sulfamoyl-benzamidine (50 mg, 0.0002 mole) in solution in DMF (1 ml). The reaction mixture was stirred at 90° C. fori 5 hours. The reaction mixture was concentrated to dryness and purify using flash chromatography to afford the title compound (60 mg). ESI-MS (ESI*): 474 (M+1H)⁺

### EXAMPLE 194

### 2-Ethyl-4-[5-(2′-fluoro-2-trifluoromethyl-biphenyl-4-yl)-[1,2,4]oxadiazol-3-yl]-benzenesulfonamide

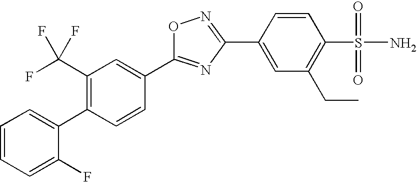

The compound is obtained using in the procedure of Example 193 using 2′-fluoro-2-trifluoromethyl-biphenyl-4-carboxylic acid instead of 2-trifluoromethyl-biphenyl-4-carboxylic acid.

ESI-MS (ESI): 478 (M−1H)⁻

### EXAMPLE 195

### 2-Ethyl-4-[5-(4-phenyl-5-trifluoromethyl-thiophen-2-yl)-[1,2,4]oxadiazol-3-yl]-benzenesulfonamide

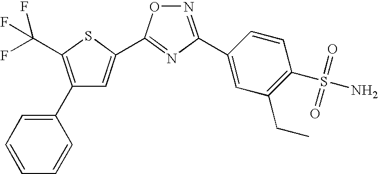

The compound is obtained using in the procedure of Example 193 using 4-phenyl-5-trifluoromethyl-thiophene-2-carboxylic acid instead of 2-trifluoromethyl-biphenyl-4-carboxylic acid.

ESI-MS (ESI): 478 (M−1H)⁻

### EXAMPLE 196

### 4-[5-(4-Cyclohexyl-3-trifluoromethyl-phenyl)-[1,2,4]oxadiazol-3-yl]-2-ethyl-benzenesulfonamide

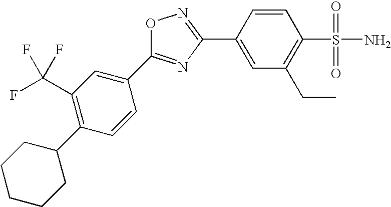

The compound is obtained using in the procedure of Example 193 using 4-cyclohexyl-3-trifluoromethyl-benzoic acid instead of 2-trifluoromethyl-biphenyl-4-carboxylic acid.

ESI-MS (ESI⁺): 480 (M+1H)⁺

### EXAMPLE 197

### 3-{4-[5-(2-Trifluoromethyl-biphenyl-4-yl)-isoxazol-3-yl]-benzenesulfonyl-amino}-propionamide

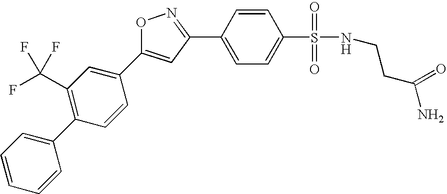

1) {4-[5-(2-trifluoromethyl-biphenyl-4-yl)-isoxazol-3-yl]-benzenesulfonyl-amino}-propionic acid is prepared as described in Example 187, starting from 4-Ethynyl-2-trifluoromethyl-biphenyl.
2) 3-{4-[5-(2-Trifluoromethyl-biphenyl-4-yl)-isoxazol-3-yl]-benzenesulfonylamino}-propionic acid (1 eq) is dissolved in DMF and subsequently N-(3-dimethylaminopropyl)-N′-ethylcarbodiimide hydrochloride (EDC.HCl; 1.5 eq), hydroxybenzotriazole (HOBt; 1.3 eq), NH₄O₂₅% in water (1.2 eq) and diisopropylethylamine (1.5 eq) are added. After 16 hours at room temperature the reaction mixture is concentrated and purified on silica gel (methylene chloride/methanol 95/5-> methylene chloride/methanol/acetic acid_{50%} 90/10/0.125 as mobile phase) resulting in pure title compound.

ESI-MS (ESI⁻): 514 (M−1H)⁻

ESI-MS (ESI⁺): 516 (M+1H)⁺

### EXAMPLE 198

### N-{4-[5-(2-Trifluoromethyl-biphenyl-4-yl)-[1,3,4]oxadiazol-2-yl]-phenyl}-succinamide

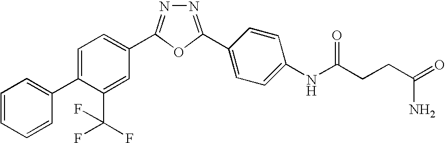

### 1) 5-(4-Nitro-phenyl)-3-(2-trifluoromethyl-biphenyl-4-yl)-[1,2,4]oxadiazole

This compound is prepared as disclosed in Example 183, starting from 2-trifluoromethyl-biphenyl-4-carboxylic acid.

### 2) 4-[5-(2-Trifluoromethyl-biphenyl-4-yl)-[1,3,4]oxadiazol-2-yl]-phenylamine

This compound is prepared as disclosed in Example 184 using the compound of step 1).

### 3) N-{4-[5-(2-Trifluoromethyl-biphenyl-4-yl)-[1,3,4]oxadiazol-2-yl]-phenyl}-succinamic acid

Endproduct (1 eq) of step 2) is dissolved in methylene chloride and 4-methylmorpholine (2 eq) and succinic anhydride (2 eq) are added. After 16 hours at room temperature pure title product is obtained after silica gel column chromatography (methylene chloride→methylene chloride/methanol 90/10 as mobile phase).

ESI-MS (ESI⁻): (M−1H)⁻; 480 (M−1H)⁻

### 4) N-{4-[5-(2-Trifluoromethyl-biphenyl-4-yl)-[1,3,4]oxadiazol-2-yl]-phenyl}-succinamide

The title compound is obtained using the same procedure as given for Example 197 in the last step (2)).

ESI-MS (ESI⁻): 479 (M−1H)⁻

ESI-MS (ESI⁺): 481 (M+1H)⁺

### EXAMPLE 199

### N-{4-[5-(2-Trifluoromethyl-biphenyl-4-yl)-[1,2,4]oxadiazol-3-yl]-phenyl}-succinamide

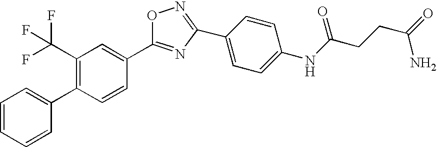

a) 2-Trifluoromethyl-biphenyl-4-carboxylic acid: it is prepared as disclosed in Example 1a).
b) N 4-Amino-N-hydroxy-benzamidine: it is prepared as disclosed in Example 1b).
c) 4-[5-(2-Trifluoromethyl-biphenyl-4-yl)-[1,2,4]oxadiazol-3-yl]-phenylamine: it is prepared as disclosed in Example 2f).
d) N-{4-[5-(2-Trifluoromethyl-biphenyl-4-yl)-[1,2,4]oxadiazol-3-yl]-phenyl}-succinamic acid: it is prepared as disclosed in Example 2 g).
e) N-{4-[5-(2-Trifluoromethyl-biphenyl-4-yl)-[1,2,4]oxadiazol-3-yl]-phenyl}-succinamide

To a solution of N-{4-[5-(2-trifluoromethyl-biphenyl-4-yl)-[1,2,4]oxadiazol-3-yl]-phenyl}-succinamic acid (1 eq) in THF is added under inert atmosphere EDC (1.3 eq) and HOBt (1.3 eq), the reaction mixture is then stirred at room temperature for 30 minutes. Then a solution of ammonium hydroxide (10 eq) is added to the reaction mixture and stirred for 4 hours at room temperature. The reaction mixture is then concentrated to dryness and purified using flash chromatography to afford N-{4-[5-(2-trifluoromethyl-biphenyl-4-yl)-[1,2,4]oxadiazol-3-yl]-phenyl}-succinamide m/z=479 (M−H)⁻.

### EXAMPLE 200

### N-Methyl-N′-{4-[5-(2-trifluoromethyl-biphenyl-4-yl)-[1,2,4]oxadiazol-3-yl]-phenyl}-succinamide

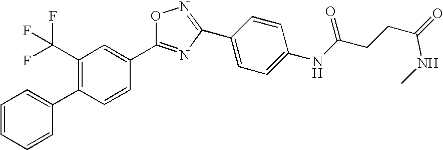

The title compound is synthesized in a similar way as disclosed in Example 199e) using a solution of methylamine in methanol instead of ammonium hydroxide in the last step, m/z=493 (M−H)⁻.

### EXAMPLE 201

### N,N-Dimethyl-N′-{4-[5-(2-trifluoromethyl-biphenyl-4-yl)-[1,2,4]oxadiazol-3-yl]phenyl}-succinamide

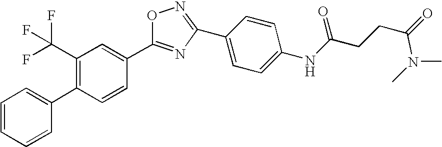

The title compound is synthesized in the similar way as in above Example 199e) using a solution of dimethylamine in methanol instead of ammonium hydroxide in the last step m/z=507 (M−H)⁻.

### EXAMPLE 202

### 3-{4-[5-(2-Trifluoromethyl-biphenyl-4-yl)-[1,3,4]oxadiazol-2-yl]benzenesulfonylamino}-propionamide

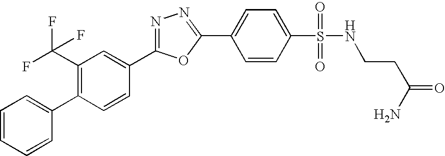

4-[5-(2-trifluoromethyl-biphenyl-4-yl)-[1,3,4]oxadiazol-2-yl]-phenylamine (1 eq) is dissolved in acetonitrile and after addition of HCl (0.55 ml; conc. 37%) and an aqueous solution of NaNO₂ (1.5 eq) the reaction mixture is kept at 8° C. for 30 minutes. To this reaction a saturated solution of SO₂ in glacial acetic acid (1 ml) and subsequently a solution of CuCl₂ in water (0.5 eq) is added. After 3 hours at room temperature the precipitate is filtered off, dissolved in methylene chloride and dried over Na₂SO₄. After removal of the solvent the title compound is used for the next step without any further purification.

ESI-MS (ESI⁺): 465 (M+1H)⁺

4-[5-(2-Trifluoromethyl-biphenyl-4-yl)-[1,3,4]oxadiazol-2-yl]-benzenesulfonyl chloride (1 eq) is dissolved in THF and H-βAla-NH₂ (3 eq), triethylamine (1 eq) and 1N NaOH (2 eq) are added. After 18 hours at room temperature the reaction is diluted with water and the pH is adjusted with 1N HCl to 3. After extraction with methylene chloride (3 times) the organic layer is dried over Na₂SO₄, filtered and concentrated. The residue is purified on silica gel (methylene chloride/methanol 8/2 as mobile phase).

ESI-MS (ESI*): 517 (M+1H)⁺

The compounds of formula I in free form or in pharmaceutically acceptable salt form, exhibit valuable pharmacological properties, e.g. as S1P1 receptor agonists, e.g. as indicated in vitro and in vivo tests and are therefore indicated for therapy.

A. In vitro

The compounds of formula I have binding affinity to individual human S1P receptors as determined in following assays:

A. In vitro: GPCR Activation Assay Measuring GTP [γ-³⁵S] Binding to Membranes Prepared from CHO Cells Expressing Human EDG Receptors

S1P₁ (EDG-1) GTP [γ-³⁵S] binding assay: Homogenized membranes are prepared from CHO cell clones stably expressing a human EDG-1 N-terminal c-myc tag. Cells are grown in suspension in two 850 cm² roller bottles for three or fours days before harvesting. The cells are centrifuged down, washed once with cold PBS, and resuspended in ≦20 ml of Buffer A (20 mM HEPES, pH 7.4, 0 mM EDTA, EDTA-free complete protease inhibitor cocktail [1 tablet/25 ml]). The cell suspension is homogenized on ice, using a Polytron homogenizer at 30000 rpm at three intervals of 15 seconds each. The homogenate is first centrifuged at 2000 rpm on a tabletop low speed centrifuge for 10 minutes. The supernatant, after passing through a cell strainer, is then re-centrifuged at 50,000×g for 25 minutes at 4° C. The pellet is resuspended into buffer B (15% glycerol, 20 mM HEPES, pH 7.4, 0.1 mM EDTA, EDTA-free complete protease inhibitor cocktail [1 tablet/10 ml]). Protein concentration of the preparation is determined using the BCA Protein Assay kit (Pierce) using BSA as standard. The membranes are aliquoted and kept frozen at −80° C.

Solutions of test compounds ranging from 10 mM to 0.01 nM are prepared in DMSO. S1P is diluted in 4% BSA solution as positive controls. The desired amount of membrane preparation is diluted with ice-cold assay buffer (20 mM HEPES, pH 7.4, 100 mM NaCl, 10 mM MgCl₂, 0.1% Fatty acid-free BSA, 5 μM GDP) and vortexed well. 2 μl or less of compound is distributed into each well of a round-bottom 96-well polystyrene assay plate, followed by addition of 100 □l of diluted membranes (3-10 ng/well) and kept on ice until the addition of hot GTPγS. [³⁵S]-GTPγS is diluted 1:1000 (v/v) with cold assay buffer and 100 μl is added into each well. The reaction is carried out at room temperature for 90 minutes before the membranes are harvested onto Perkin-Elmer Unifilter® GF/B-96 filter plate using a Packard Filtermate Harvester. After several washes with wash buffer (20 mM HEPES, pH 7.4, 100 mM NaCl, 10 mM MgCl₂), and a rinse with 95% ethanol, the filter is dried in a 37° C. oven for 30 minutes. MicroScint-20 is added and the plate sealed for scintillation counting on TopCount. EC₅₀ values are obtained by fitting the GTP [γ-³⁵S] binding curves (raw data) with the dose response curve-fitting tool of GraphPad Prism. Six or twelve different concentrations are used to generate a concentration response curve (using three data points per concentration).

S1P3,-5,-6 and -8 GTP [γ-³⁵S] binding assays are carried out in a comparable manner to the S1P1 GTP [γ-³⁵S] binding assay using membranes from CHO cells stably expressing c-terminal c-myc tagged or untagged receptors. For each membrane preparation, titration experiments are first run with S1P control to determine the optimal amount of membranes to be added per assay well.

Compounds of formula I are tested according to the above assay and show binding affinity to to S1P receptors, e.g. S1P1 receptors with an EC₅₀<1 μM. More particularly, compounds of formula I exhibit selectivity for the S1P1 receptor. For example, Compounds of Examples 45, 54, 145, 194 and 196 have an EC₅₀<1 nM in the above S1P1 receptor binding assay and are at least 20 fold selective for S1P1 receptor compared to S1P3 receptor, and at least 20 fold selective for S1P1 receptor compared to S1P8 receptor.

### B. In Vitro: FLIPR Calcium Flux Assay

Compounds of formula I are tested for agonist activity on S1P1, S1P3, S1P5, and S1P6 with a FLIPR calcium flux assay. Briefly, CHO cells expressing an S1P receptor are maintained in F-12K medium (ATCC), containing 5% FBS, with 500 μg/ml of G418. Prior to the assay, the cells are plated in 384 black clear bottom plates at the density of 10,000 cells/well/25 μl in the medium of F-12K containing 1% FBS. The second day, the cells are washed three times (25 μl/each) with washing buffer. About 25 μl of dye are added to each well and incubated for 1 hour at 37° C. and 5% CO₂. The cells are then washed four times with washing buffer (25 μl/each). The calcium flux is assayed after adding 25 μl of SEW2871 (published by Rosen et al., used as reference) solution to each well of cells. The same assay is performed with cells expressing each of the different S1P receptors. Titration in the FLIPR calcium flux assay is recorded over a 3-minute interval, and quantitated as maximal peak height percentage response relative to S1P-1 activation. The compounds of formula I are active in this assay at a concentration of from 10⁻¹² and 3.10⁻⁵ nM.

### C. In Vivo: Screening Assays for Measurement of Blood Lymphocyte Depletion

Measurement of circulating lymphocytes: Compounds to be tested are dissolved in DMSO/PEG200 and further diluted with deionized water. Rats (Lewis strain, female, 6-12 weeks old) are administered 1 mg/kg of compound to be tested in 4 ml/kg vehicle (max. 2% DMSO/max. 2% PEG200/water) via per os application. DMSO/PEG200/water and FTY720 (0.3 mg/kg) are included as negative and positive controls, respectively.

Blood is collected from the sublingual vein 2, 6, 24 and 48 hours after administration under short isoflurane anesthesia. Whole blood samples are subjected to hematology analysis. Peripheral lymphocyte counts are determined using an automated analyzer. Subpopulations of peripheral blood lymphocytes are stained by fluorochrome-conjugated specific antibodies and analyzed using a fluorescent activating cell sorter (Facscalibur). Two rats are used to assess the lymphocyte depletion activity of each compound screened. The result is an EDso, which is defined as the effective dose required to display 50% of blood lymphocyte depletion. Compounds of formula I are tested according to the above assay and have an ED50 of less than 10 mg/kg.

The compounds of formula I are, therefore, useful in the treatment and/or prevention of diseases or disorders mediated by lymphocytes interactions, e.g. in transplantation, such as acute or chronic rejection of cell, tissue or organ alio- or xenografts or delayed graft function, graft versus host disease, autoimmune diseases, e.g. rheumatoid arthritis, systemic lupus erythematosus, hashimoto's thyroidis, multiple sclerosis, myasthenia gravis, diabetes type I or II and the disorders associated therewith, vasculitis, pernicious anemia, Sjoegren syndrome, uveitis, psoriasis, Graves opthalmopathy, alopecia greata and others, allergic diseases, e.g. allergic asthma, atopic dermatitis, allergic rhinitis/conjunctivitis, allergic contact dermatitis, inflammatory diseases optionally with underlying aberrant reactions, e.g. inflammatory bowel disease, Crohn's disease or ulcerative colitis, intrinsic asthma, inflammatory lung injury, inflammatory liver injury, inflammatory glomerular injury, atherosclerosis, osteoarthritis, irritant contact dermatitis and further eczematous dermatitises, seborrhoeic dermatitis, cutaneous manifestations of immunologically-mediated disorders, inflammatory eye disease, keratoconjunctivitis, myocarditis or hepatitis, e.g. acute or chronic hepatitis, ischemia/reperfusion injury, e.g. myocardial infarction, stroke, gut ischemia, renal failure or hemorrhage shock, traumatic shock, cancer, e.g. breast cancer, T cell lymphomas or T cell leukemias, nephrotic syndrome, infectious diseases, e.g. toxic shock (e.g. superantigen induced), septic shock, adult respiratory distress syndrome or viral infections, e.g. AIDS, viral hepatitis, e.g. hepatitis B or C, chronic bacterial infection, or neurodegenerative diseases, e.g. Alzheimer disease, amyotrophic lateral sclerosis or senile dementia. Examples of cell, tissue or solid organ transplants include e.g. pancreatic islets, stem cells, bone marrow, corneal tissue, neuronal tissue, heart, lung, combined heart-lung, kidney, liver, bowel, pancreas, trachea or oesophagus. For the above uses the required dosage will of course vary depending on the mode of administration, the particular condition to be treated and the effect desired.

In general, satisfactory results are indicated to be obtained systemically at daily dosages of from about 0.03 to 5.0 mg/kg per body weight. An indicated daily dosage in the larger mammal, e.g. humans, is in the range from about 0.5 mg to about 500 mg, conveniently administered, for example, in divided doses up to four times a day or in retard form. Suitable unit dosage forms for oral administration comprise from ca. 0.1 to 50 mg active ingredient.

The compounds of formula I may be administered by any conventional route, in particular enterally, e.g. orally, e.g. in the form of tablets or capsules, or parenterally, e.g. in the form of injectable solutions or suspensions, topically, e.g. in the form of lotions, gels, ointments or creams, or in a nasal or a suppository form. Pharmaceutical compositions comprising a compound of formula I in free form or in pharmaceutically acceptable salt form in association with at least one pharmaceutical acceptable carrier or diluent may be manufactured in conventional manner by mixing with a pharmaceutically acceptable carrier or diluent.

The compounds of formula I may be administered in free form or in pharmaceutically acceptable salt form e.g. as indicated above. Such salts may be prepared in conventional manner and exhibit the same order of activity as the free compounds.

In accordance with the foregoing the present invention further provides:
1.1 A method for preventing or treating disorders or diseases mediated by lymphocytes, e.g. such as indicated above, in a subject in need of such treatment, which method comprises administering to said subject an effective amount of a compound of formula I or a pharmaceutically acceptable salt thereof;
1.2 A method for preventing or treating acute or chronic transplant rejection or T-cell mediated inflammatory or autoimmune diseases, e.g. as indicated above, in a subject in need of such treatment, which method comprises administering to said subject an effective amount of a compound of formula I or a pharmaceutically acceptable salt thereof;
2. A compound of formula I, in free form or in a pharmaceutically acceptable salt form for use as a pharmaceutical, e.g. in any of the methods as indicated under 1.1 or 1.2 above.
3. A pharmaceutical composition, e.g. for use in any of the methods as in 1.1 or 1.2 above comprising a compound of formula I in free form or pharmaceutically acceptable salt form in association with a pharmaceutically acceptable diluent or carrier therefore.
4. A compound of formula I or a pharmaceutically acceptable salt thereof for use in the preparation of a pharmaceutical composition for use in any of the method as in 1.1 or 1.2 above.

The compounds of formula I may be administered as the sole active ingredient or in conjunction with, e.g. as an adjuvant to, other drugs e.g. immunosuppressive or immunomodulating agents or other anti-inflammatory agents, e.g. for the treatment or prevention of alio- or xenograft acute or chronic rejection or inflammatory or autoimmune disorders, or a chemotherapeutic agent, e.g a malignant cell anti-proliferative agent. For example, the compounds of formula I may be used in combination with a calcineurin inhibitor, e.g. cyclosporin A or FK 506; a mTOR inhibitor, e.g. rapamycin, 40-O-(2-hydroxyethyl)-rapamycin, CCI779, ABT578, AP23573, biolimus-7 or biolimus-9; an ascomycin having immuno-suppressive properties, e.g. ABT281, ASM981, etc.; corticosteroids; cyclophosphamide; azathioprene; methotrexate; leflunomide; mizoribine; mycophenolic acid or salt; mycophenolate mofetil; 15-deoxyspergualine or an immunosuppressive homologue, analogue or derivative thereof; a PKC inhibitor, e.g. as disclosed in WO 02/38561 or WO 03/82859, e.g. the compound of Example 56 or 70; a JAK3 kinase inhibitor, e.g. N-benzyl-3,4-dihydroxy-benzylidene-cyanoacetamide α-cyano-(3,4-dihydroxy)-]N-benzylcinnamamide (Tyrphostin AG 490), prodigiosin 25-C (PNU156804), [4-(4′-hydroxyphenyl)-amino-6,7-dimethoxyquinazoline] (WH1-P131), [4-(3′-bromo-4′-hydroxyphenyl)-amino-6,7-dimethoxyquinazoline] (WHI-P154), [4-(3′,5′-dibromo-4′-hydroxylphenyl)-amino-6,7-dimethoxyquinazoline] WHI-P97, KRX-211, 3-{(3R,4R)-4-methyl-3-[methyl-(7H-pyrrolo[2,3-d]pyrimidin-4-yl)-amino]-piperidin-1-yl}-3-oxo-propionitrile, in free form or in a pharmaceutically acceptable salt form, e.g. mono-citrate (also called CP-690,550), or a compound as disclosed in WO 04/052359 or WO 05/066156; immunosuppressive monoclonal antibodies, e.g., monoclonal antibodies to leukocyte receptors, e.g., MHC, CD2, CD3, CD4, CD7, CD8, CD25, CD28, CD40, CD45. CD52, CD58. CD80, CD86 or their ligands; other immunomodulatory compounds, e.g. a recombinant binding molecule having at least a portion of the extracellular domain of CTLA4 or a mutant thereof, e.g. an at least extracellular portion of CTLA4 or a mutant thereof joined to a non-CTLA4 protein sequence, e.g. CTLA4Ig (for ex. designated ATCC 68629) or a mutant thereof, e.g. LEA29Y; adhesion molecule inhibitors, e.g. LFA-1 antagonists, ICAM-1 or -3 antagonists, VCAM-4 antagonists or VLA-4 antagonists; or a chemotherapeutic agent, e.g. paclitaxel, gemcitabine, cisplatinum, doxorubicin or 5-fluorouracil; or an anti-infectious agent.

Where the compounds of formula I are administered in conjunction with other immunosuppressive/immunomodulatory, anti-inflammatory, chemotherapeutic or anti-infectious therapy, dosages of the co-administered immunosuppressant, immunomodulatory, anti-inflammatory, chemotherapeutic or anti-infectious compound will of course vary depending on the type of co-drug employed, e.g. whether it is a steroid or a calcineurin inhibitor, on the specific drug employed, on the condition being treated and so forth. In accordance with the foregoing the present, invention provides in a yet further aspect:
5. A method as defined above comprising co-administration, e.g. concomitantly or in sequence, of a therapeutically effective non-toxic amount of a compound of formula I and at least a second drug substance, e.g. an immunosuppressant, immunomodulatory, anti-inflammatory or chemotherapeutic drug, e.g. as indicated above.
6. A pharmaceutical combination, e.g. a kit, comprising a) a first agent which is a compound of formula I as disclosed herein, in free form or in pharmaceutically acceptable salt form, and b) at least one co-agent, e.g. an immunosuppressant, immunomodulatory, anti-inflammatory, chemotherapeutic or anti-infectious agent. The kit may comprise instructions for its administration.

The terms “co-administration” or “combined administration” or the like as utilized herein are meant to encompass administration of the selected therapeutic agents to a single patient, and are intended to include treatment regimens in which the agents are not necessarily administered by the same route of administration or at the same time.

The term “pharmaceutical combination” as used herein means a product that results from the mixing or combining of more than one active ingredient and includes both fixed and non-fixed combinations of the active ingredients. The term “fixed combination” means that the active ingredients, e.g. a compound of formula I and a co-agent, are both administered to a patient simultaneously in the form of a single entity or dosage. The term “non-fixed combination” means that the active ingredients, e.g. a compound of formula I and a co-agent, are both administered to a patient as separate entities either simultaneously, concurrently or sequentially with no specific time limits, wherein such administration provides therapeutically effective levels of the 2 compounds in the body of the patient. The latter also applies to cocktail therapy, e.g. the administration of 3 or more active ingredients.

1. A compound of formula I 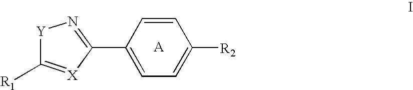
wherein
either X is —N═ and Y is —O—; or X is —O— and Y is —N═; or X is CH and Y is O;
is substituted biphenylyl, 4-phenoxy-phenyl or 4-(phenyl-C₁₋₄alkoxy)-phenyl wherein at least one of the phenyl groups is monosubstituted; phenyl substituted by one or more substituents selected from halogen, nitrile, C₁₋₈alkyl, haloC₁₋₈alkyl, C₁₋₈alkoxy, haloC₁₋₈alkoxy, C₁₋₈alkoxy-C₁₋₈alkoxy, C₁₋₈alkyl-C₁₋₈alkoxy, C₁₋₈alkyl-haloC₁₋₈alkoxy, haloC₁₋₈alkyl-C₁₋₈alkoxy, haloC₁₋₈alkyl-haloC₁₋₈alkoxy, haloC₁₋₈alkoxy-C₁₋₈alkoxy, C₁₋₈alkoxy-haloC₁₋₈alkoxy, haloC₁₋₈alkoxy-1-haloC₁₋₈alkoxy, C₁₋₈alkoxy-C₁₋₈alkyl, haloC₁₋₈alkoxy-C₁₋₈alkyl, C₁₋₈alkoxy-haloC₁₋₈alkyl, haloC₁₋₈alkoxy-haloC₁₋₆alkyl, C₁₋₈alkenyloxy, C₂₋₆alkynyloxy, C₃₋₆cycloalkyl, C₃₋₆cycloalkyl-C₁₋₄alkyl, C₃₋₆cycloalkyl-C₁₋₄alkoxy, C₃₋₆cycloalkyl-oxy, phenyl-C₁₋₄alkoxy and heterocyclic-C₁₋₄alkoxy; or substituted 5 or 6-membered heteroaryl;
R₂ is C₁₋₆ alkyl optionally substituted by halogen, OH, NH₂, C₁₋₄alkoxy or C₁₋₄-alkylcarbonyloxy; amino; carboxy; sulfamoyl; carbamoyl; or HN—CO—C₁₋₄-alkyl; or
R₂ is R₃—R₄—COOH or R₃—R₄—CONR₅R₆
wherein R₃ is SO₂—NH; SO₂—N(C₁₋₄alkyl); CO—NH; CO—N(C₁₋₄alkyl); CH₂—O; NH—CO; or
N(C₁₋₄-alkyl)CO; and R₄ is C₁₋₆alkylene optionally interrupted by O, S or C═CH₂ or optionally substituted phenylene or C₃₋₆cycloalkylene; and each of R₅ and R₆, independently, is hydrogen or C₁₋₆alkyl or R₅ and R₆ together with the nitrogen atom to which they are bound form a heterocyclic residue, and
ring A may optionally be substituted,
provided that when Y is O, X is —N═ or —CH═ and R₂ is SO₂NH—R₄—COOH wherein R₄ is branched C₁₋₆alkylene, then
i. R₁ is other than phenyl either monosubstituted by halogen, C₁₋₈alkyl, C₁₋₈alkoxy, haloC₁₋₈alkyl or haloC₁₋₈alkoxy, or disubstituted by one or two substituents selected from halogen, C₁₋₈alkyl and C₁₋₈alkoxy; or
ii. is other than monosubstituted thienyl or furyl
or a physiologically hydrolysable derivative thereof, a salt, hydrate and/or solvate thereof.

2. A compound according to claim 1 wherein R₁ is substituted biphenylyl, 4-phenoxy-phenyl or 4-(phenyl-C₁₋₄alkoxy)-phenyl wherein at least one of the phenyl groups is monosubstituted.

3. A compound according to claim 1 wherein R₂ is C₁₋₆alkyl optionally substituted by halogen, OH, NH₂, C₁₋₄alkoxy or C₁₋₄alkylcarbonyloxy; amino; carboxy; sulfamoyl; carbamoyl; or HN—CO—C₁₋₄-alkyl.

4. A process for the production of a compound of formula I according to claim 1 which process comprises
a) for the production of a compound of formula I wherein X is —N↑ and Y is O and R₂ is as defined above, reacting a compound of formula II 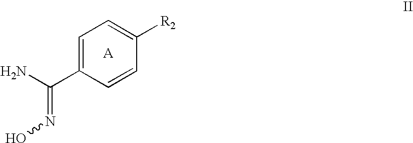
wherein ring A and R₂ are as defined in claim 1
with a compound of formula III 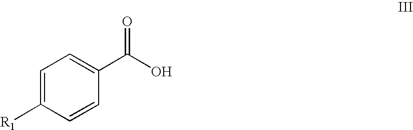
wherein R₁ is as defined above or a functional derivative thereof; or
b) for the production of a compound of formula I wherein X is —N═ and Y is O and R₂ is R₃—R₄—COOH or R₃—R₄—CONR₅R₆ wherein R₃ is NH—CO or N(C₁₋₄alkyl)CO and R₄, R₅ and R₆ is as defined in claim 1, reacting a compound of formula IV 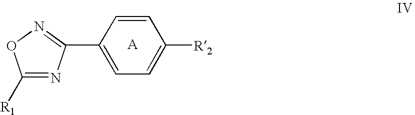
wherein R₁ and ring A are as defined in claim 1 and R′₂ is NH₂ or NH(C₁₋₄alkyl), with an acylating agent or by following a Sandmeyer reaction; or
c) for the production of a compound of formula I wherein Y is —N═ and X is O, cyclizing in the presence of a Burgess reagent, a compound of formula V 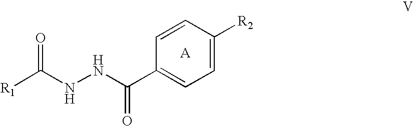
wherein R₁, R₂ and ring A are as defined in claim 1; or
d) for the production of a compound of formula I wherein Y is O and X is CH, reacting a compound of formula VI 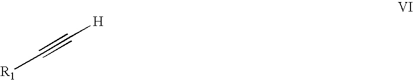
wherein R₁ is as defined in claim 1, with a compound of formula VII 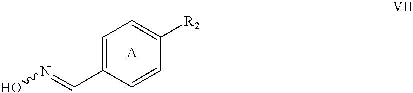
wherein R₂ is as defined in claim 1; or
e) converting a compound of formula I into another compound of formula I,
and recovering the resulting compound of formula I in free form or in form of a salt, and, where required converting the compound of formula I obtained in free form into the desired salt form or vice versa.

5. A compound of formula I according to claim 1 or a pharmaceutically acceptable salt thereof for use as a pharmaceutical.

6. A pharmaceutical composition comprising a compound of formula I according to claim 1 or a pharmaceutically acceptable salt thereof, in association with a pharmaceutically acceptable diluent or carrier therefore.

7. A compound of formula I according to claim 1 or a pharmaceutically acceptable salt thereof for use in the preparation of a pharmaceutical composition for use in preventing or treating disorders or diseases mediated by lymphocytes.

8. A pharmaceutical combination comprising a) a first agent which is a compound of formula I according to claim 1, in free form or in pharmaceutically acceptable salt form, and b) at least one co-agent which is an immunosuppressant, immunomodulatory, anti-inflammatory, chemotherapeutic or anti-infectious agent.

9. A method for preventing or treating disorders or diseases mediated by lymphocytes, in a subject in need of such treatment, which method comprises administering to said subject an effective amount of a compound of formula I according to claim 1 or a pharmaceutically acceptable salt thereof.
